(19) United States
(12) Patent Application Publication
(10) Pub. No.: US 2010/0136130 A1
(43) Pub. Date: Jun. 3, 2010

(54) Preparation for the Controlled Release of Bioactive Natural Substances

(75) Inventors: Matthias Seiler, Griesheim (DE); Martin Haneke, Rosenheim (DE); Henning Marckmann, Buchloe (DE); Stephan Pilz, Fellbach (DE); Muhammad Irfan, Hanau (DE); Saskia Klee-Laquai, Hilden (DE); Geoffrey Hills, Essen (DE); Mike Farwick, Essen (DE); Peter Lersch, Dinslaken (DE); Axel Kobus, Langen (DE)

Correspondence Address: LAW OFFICE OF MICHAEL A. SANZO, LLC, 15400 CALHOUN DR., SUITE 125, ROCKVILLE, MD, 20855, US

(73) Assignee: EVONIK DEGUSSA GMBH, Essen (DE)

(21) Appl. No.: 12/596,401

(22) PCT Filed: Apr. 15, 2008
(86) PCT No.: PCT/EP2008/054536

§ 371 Date: Jan. 23, 2010

(30) Foreign Application Priority Data
Apr. 18, 2007 (EP) EP07106432

(51) Int. Cl. A61K 36/736; A61K 9/14; A61P 43/00

(52) U.S. Cl. 424/501; 424/735

(57) ABSTRACT

The present invention relates to a preparation comprising at least one encapsulation material and at least one bioactive natural substance, which bioactive natural substance can be released from the preparation in a controlled manner, wherein the encapsulation material comprises at least one glyceride with a melting point of at least 35° C. and additionally at least one polymer with polyester units, which has a melting temperature of at least 30° C. and a viscosity in the range from 50 mPa*s to 250 Pa*s, measured by means of rotational viscometry at 110° C. The present invention further describes processes for producing the preparation of the invention, as well as preferred uses.

The present invention relates to preparations for controlled release of bioactive natural substances and to processes for production of and to the use of these preparations.

Many bioactive natural substances, for example plant extracts, vitamins or oils which contain a high proportion of unsaturated fatty acids, are very oxidation-sensitive and therefore have to be stored at low temperatures or under inert gas atmosphere. However, these storage conditions can be maintained in many cases only to a very limited degree. For this reason, many of these substances are converted to preparations which enable simple storage. In addition, these preparations in many cases allow controlled release of these active ingredients at an intended location or over a particular time.

In general, these preparations in many cases are described as encapsulated systems which comprise an active ingredient and an encapsulation material which exerts a protective function or whose properties can be used to control the release profile.

The encapsulation materials used are in many cases polymers, for example hyperbranched polymers. The use of hyperbranched polymers as a carrier substance for medicaments is detailed, for example, in WO 2004/072153. According to this publication, the carrier molecule enables retarded release and facilitated transport of the medicaments into the cells. In this connection, especially modified dendrimers are detailed, which have nitrogen-comprising groups.

In addition, publication WO 00/065024 describes polymers for encapsulating hydrophobic molecules. In this case, a multitude of hydrophobic radicals are bonded to a polyol core, the polymer obtained subsequently being converted by polyalkylene oxides in order to obtain a water-soluble polymer.

In addition, publication WO 2005/034909 describes compositions comprising a hyperbranched polymer coupled to a biologically active radical.

In addition, publication WO 03/037383 describes preparations which comprise hyperbranched polymers. The hyperbranched polymers detailed are especially polyamidoamines or polypropyleneamines.

In addition, hyperbranched polymers are described in the publication WO 00/06267, the hyperbranched polymers detailed being especially polyetherimides.

In addition, preparations which comprise dendritic polymers and active pharmaceutical ingredients are detailed in WO 03/033027, the dendrimer comprising cationic groups.

In addition, the use of hyperbranched polymers for controlled release of active ingredients is described by Zou et al. Macromol. Biosci. 5 (2005) 662-668. In this case, hyperbranched polymers are provided with ionic groups.

In addition, publications U.S. Pat. No. 6,379,683 and EP 1 034 839 B1 describe nanocapsules which comprise hyperbranched polymers. A disadvantage here is especially that the nanocapsules cannot be isolated from the dispersion and processed further. Furthermore, the nanocapsules are produced using organic solvents, which in many cases cannot be removed completely from the dispersion.

Document U.S. Pat. No. 6,432,423 describes hyperbranched polymers which may comprise polyester units. These polymers serve especially as film formers in cosmetic compositions which may contain fats. However, the melting point of the fats is not described. In addition, this polymer is detailed merely as a constituent of a conventional mixture, without this achieving controlled release of the active ingredients.

Polymers similar to the polymers described in U.S. Pat. No. 6,432,423 are additionally detailed in U.S. Pat. No. 6,475,495. According to this publication, these polymers can be used especially as gelating agents in cosmetic compositions. However, no preparations which enable controlled release of active ingredients are described.

Cosmetic compositions which comprise hyperbranched polymers are also detailed in US 2006/0030686. In this context, these polymers serve especially as formulating auxiliaries. Preparations which allow controlled active ingredient release are, however, not described.

Preparations by which controlled release of active ingredients is achieved are, for example, the subject of publication WO 2006/047714. These preparations comprise especially an active ingredient bound covalently to a polymer or oligomer. The active ingredient is released especially through an enzymatic cleavage of this covalent bond. This can advantageously release the active ingredient in a controlled manner at a given site of action. A disadvantage of these systems is, however, that the active ingredient must first be bonded to a polymer. This limits the application of this process. Furthermore, the storability of the active ingredients is improved only insignificantly as a result. In addition, near-natural active ingredient combinations, for example plant extracts, can be converted to a corresponding preparation in natural composition only with very great difficulty.

Publication WO 2004/028269 describes polymers which can be used in biodegradable chewing gums. These polymers, which may comprise especially polyester units, have a particular degree of branching. However, the controlled release of active ingredients is not the subject of this publication.

The thesis by S. Suttiruengwong “Silica Aerogels and Hyperbranched Polymers as Drug Delivery Systems”, Erlangen 2005, describes encapsulated systems which may comprise hyperbranched polymers.

Furthermore, document US 2004/016394 describes cosmetic compositions which may comprise hyperbranched polymers. However, the properties of these hyperbranched polymers, especially the molecular weight, the degree of branching, the hydroxyl number or the melting point, are not stated explicitly.

Accordingly, it is recorded that many known preparations comprise an encapsulation material, for example a hyperbranched polymer, and an active ingredient. However, it is always desirable to provide very advantageous preparations. In addition, the cost and complexity for the production of the hyperbranched polymers described above is relatively high. Accordingly, these polymers are comparatively expensive.

Furthermore, the handling of the prior art preparations is difficult, since they are present as dispersions in many cases and cannot be isolated therefrom. Accordingly, these preparations can be incorporated into end products only in a very limited and technically demanding manner.

Furthermore, organic solvents are used in many cases to produce the prior art preparations detailed above. The end user, however, desires near-natural products which do not have a residual content of these compounds.

In view of the prior art specified and discussed herein, it was an object of the present invention to provide preparations which have an outstanding profile of properties.

The profile of properties comprises especially the means of controlling the release of the bioactive natural substance. In one aspect of the present invention, the inventive preparations should be able to release the bioactive natural substance in a selected medium over a very long period, in the course of which the release rate should preferably remain constant. In a further embodiment of the present invention, the release should proceed in a controlled manner within a short period. In this context, one object can be considered that of providing a preparation in which the release of the active ingredient can be controlled in a very simple and reliable manner.

It was a further object of the present invention to provide preparations which comprise a particularly high content of bioactive natural substance.

Furthermore, the preparation should exhibit a particularly high stability, as a result of which especially sensitive natural substances can be stored over a particularly long period, without the properties of the natural substance being altered significantly. At the same time, the preparations should likewise have a high shear stability, such that simple and problem-free processing of the preparations is possible.

It was a further object to provide preparations which can be produced in a simple and inexpensive manner.

These objects and further objects which are not stated explicitly but can be derived as a matter of course from the context discussed herein or inevitably result therefrom are achieved by the preparations described in claim 1.

Appropriate modifications of these preparations are protected in the subclaims which refer back to claim 1.

The present invention accordingly provides a preparation comprising an encapsulation material and at least one bioactive natural substance, which bioactive natural substance can be released from the preparation in a controlled manner, which is characterized in that the encapsulation material comprises at least one glyceride with a melting point of at least 35° C. and additionally at least one polymer with polyester units, said polymer with polyester units having a melting temperature of at least 35° C. and a viscosity in the range from 50 mPa*s to 250 Pa*s, measured by means of rotational viscometry at 110° C.

By virtue of the inventive measures, it is surprisingly possible to provide preparations with an outstanding profile of properties, which can be obtained in a particularly simple and inexpensive manner, especially without use of organic solvents.

The inventive preparations may especially have an outstanding release profile of the bioactive natural substance, it being possible to achieve either a release over a particularly long period or a quick release after actuation of a trigger mechanism.

In addition, the preparations may have a high shear stability. This allows the preparations obtainable in accordance with the invention to be processed in a particularly simple and problem-free manner. Moreover, the preparations can be adjusted to particular needs in a particularly simple manner. For instance, preparations may have a wide variety of different release mechanisms. These include mechanisms based on an enzymatic degradation of the encapsulation material or a pH-selective opening of the encapsulation material; temperature- or solvent-controlled processes, action of energy on the preparations, for example irradiation of particles with electromagnetic radiation, irradiation with ultrasound and/or action of shear forces. In addition, the preparations may have a high proportion of bioactive natural substance.

Furthermore, the preparation detailed in the present document enables particularly stable storage of sensitive natural substances. Accordingly, sensitive substances can be stored in a reliable and simple manner.

Moreover, the inventive preparations are surprisingly stable, such that they can be stored over a long period without degradation. In addition, the preparations can be processed without any problem owing to the high shear stability. A particular advantage arises especially from the fact that the preparations are solid at room temperature, especially at 25° C., such that this solid can be incorporated into many end products without process steps which are complex and difficult to control.

The preparations of the present invention can in many cases be obtained in a particularly simple and inexpensive manner. Furthermore, the inventive preparations are not hazardous to health. In this context, it is especially advantageous that the inventive preparations are obtainable without the use of organic solvents. It is therefore possible to obtain very near-natural preparations which do not contain a residual content of organic solvents.

The term “encapsulation material” refers especially to a mixture which comprises at least one glyceride with a melting point of at least 35° C. and additionally at least one polymer with polyester units, said polymer with polyester units having a melting temperature of at least 35° C. and a viscosity in the range from 50 mPa*s to 100 Pa*s. The encapsulation material serves especially for protection of the bioactive natural substance. Accordingly, the encapsulation material preferably has a high stability to oxidation. Of particular interest in this context are especially materials which are enzymatically degradable. This makes it possible especially to provide a mechanism which enables a controlled release of the active ingredients on human skin.

The encapsulation material to be used in accordance with the invention comprises at least one glyceride which has a melting point of at least 35° C., preferably at least 40° C. Of particular interest are especially glycerides which have a melting point in the range from 45 to 80° C., more preferably 50 to 65° C.

Appropriate embodiments of the encapsulation material to be used in accordance with the invention comprise especially a glyceride which has a dynamic viscosity in the range from 5 to 200 mPa*s, preferably 10 to 50 mPa*s and more preferably 15 to 22 mPa*s at 70° C., measured to DIN EN ISO 3219.

In the present context, the term “glyceride” denotes an ester of a carboxylic acid with glycerol (propane-1,2,3-triol). Two or three of the hydroxyl groups of the glycerol may preferably be esterified with two or three carboxylic acids which have 8 to 35, especially 8 to 30, preferably 12 to 24 and more preferably 14 to 20 carbon atoms. Of particular interest are especially triglyceryl esters which have three groups derived from carboxylic acids having 12 to 24 and more preferably 14 to 20 carbon atoms.

Preferred triglyceryl esters have especially the formula (I) in which the R1, R2 and R3 radicals are each independently a hydrocarbon radical having 7 to 34, especially 7 to 29, preferably 11 to 23 and more preferably 13 to 19 carbon atoms.

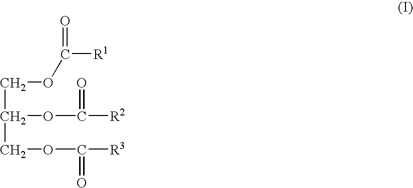

In the present context, hydrocarbon radicals denote especially saturated and/or unsaturated radicals which consist preferably of carbon and hydrogen. These radicals may be cyclic, linear or branched. They include especially alkyl radicals and alkenyl radicals, where the alkenyl radicals may comprise one, two, three or more carbon-carbon double bonds.

The preferred alkyl radicals include especially the heptyl, octyl, 1,1,3,3-tetramethylbutyl, nonyl, decyl, undecyl, dodecyl, tridecyl, tetradecyl, pentadecyl, hexadecyl, heptadecyl, octadecyl, nonadecyl, eicosyl and the cetyleicosyl group.

Examples of alkenyl radicals with one carbon-carbon double bond include the heptenyl, octenyl, nonenyl, decenyl, undecenyl, dodecenyl, tridecenyl, tetradecenyl, pentadecenyl, hexadecenyl, heptadecenyl, octadecenyl, nonadecenyl, eicosenyl and the cetyleicosenyl group.

The hydrocarbon radicals detailed above may have substituents and/or heteroatoms. These include especially groups which comprise oxygen, nitrogen and/or sulfur, for example hydroxyl groups, thiol groups or amino groups. However, the proportion of these groups should be sufficiently low that the properties of the glycerides are not adversely affected.

The preferred saturated carboxylic acids from which the glycerides are derived include octanoic acid, decanoic acid, dodecanoic acid, tetradecanoic acid, hexadecanoic acid, heptadecanoic acid, octadecanoic acid, eicosanoic acid, docosanoic acid, tetracosanoic acid, hexacosanoic acid, octacosanoic acid, triacontanoic acid, tetratriacontanoic acid and pentatriacontanoic acid, more preferably eicosanoic acid and docosanoic acid.

The properties of the glycerides depend especially on the type and the amount of the fatty acids present in the glycerides. Accordingly, the proportion of the long-chain saturated fatty acids is very high compared to the proportions which occur in many naturally occurring oils and fats. According to the invention, it is possible to use a monoglyceride which was obtained by esterifying glycerol with a particularly long-chain fatty acid. In addition, it is possible to use di- or triglycerides which have a correspondingly high proportion of carboxylic acids which have 12 to 36 and preferably 14 to 24 carbon atoms.

Of particular interest are especially triglycerides which have a high proportion of stearic acid and/or palmitic acid. It is possible with preference to use especially triglycerides which have at least 10% by weight, preferably at least 20% by weight and most preferably at least 30% by weight of stearic acid and/or palmitic acid radicals, based on the total content of fatty acids. In a preferred embodiment, especially triglycerides which comprise stearic acid and palmitic acid groups are used. The weight ratio of stearic acid to palmitic acid is preferably in the range from 10:1 to 1:1, more preferably 4:1 to 1.5:1. In a particularly appropriate embodiment, it is especially possible to use a triglyceride whose fatty acid spectrum comprises preferably 50 to 90% by weight, more preferably 60 to 80% by weight and most preferably about 70% by weight of stearic acid, and preferably 10 to 50% by weight, more preferably 20 to 40% by weight and most preferably about 30% by weight of palmitic acid.

The oxidation stability of the glycerides used as the encapsulation material depends inter alia on the proportion of unsaturated carboxylic acids. The lower this proportion, the higher the oxidation stability. A glyceride for use with preference preferably has an iodine number less than or equal to 50, particularly less than or equal to 20 and more preferably less than or equal to 10. The iodine number can be determined especially according to DIN EN 14111.

In addition to a synthesis of the glycerides detailed above by esterifying glycerol with appropriate carboxylic acids, these glycerides can also be obtained from natural sources, especially from plants. It is thus possible to use especially triglycerides which are obtainable commercially from Goldschmidt GmbH, Essen under the Tegin® trade name. In addition, it is possible to use suitable triglycerides which are obtainable from Cognis GmbH & Co. KG under the Edenor® name, it being possible to use especially Edenor® NHTi V. The glyceride for use as an encapsulation material in accordance with the invention differs from the bioactive natural substances to be released. In general, the natural substances to be released are oxidation-sensitive or otherwise unstable. Accordingly, the glyceride for use as the encapsulation material can be distinguished from the bioactive natural substance by the oxidation sensitivity, which can be determined, for example, by the iodine number (grams of iodine which can be added onto the double bonds of 100 g of natural substance). Of particular interest are especially preparations whose bioactive natural substance has an iodine number which is at least 5 greater than the iodine number of the glyceride for use as the encapsulation material. This iodine number difference is preferably at least 10, more preferably at least 20.

The molecular weight of the glycerides for use with preference as the encapsulation material is not critical per se. In general, in many cases, glycerides with a molecular weight in the range from 200 to 1600 g/mol, preferably 400 to 1500 g/mol and more preferably 500 to 1200 g/mol are used.

The proportion of glyceride based on the weight of the preparation is preferably in the range from 1 to 99.5% by weight, more preferably 10 to 80% by weight and most preferably 20 to 70% by weight.

The encapsulation material comprises besides at least one glyceride additionally a polymer which comprises polyester units. In the context of the present invention, the term “polymer with polyester units” denotes a macromolecular compound which comprises units which can form polyesters. These include especially units derived from diols and dicarboxylic acids, and units derived from compounds with at least one carboxylic acid group and at least one hydroxyl group. The proportion of units which can form polyesters, based on the weight of the polymer, is preferably at least 10% by weight, more preferably at least 30% by weight.

The polymer with polyester units has a melting temperature of at least 30° C., preferably at least 35° C. Especially appropriate are polymers with polyester units which have a melting temperature range from 30° C. to 90° C., more preferably 35° C. to 70° C. and most preferably 40° C. to 65° C. The melting temperature can be determined by means of differential scanning calorimetry (DSC), for example with the Mettler DSC 27 HP apparatus and a heating rate of 10° C./min.

The polymer with polyester units present in the encapsulation material has a viscosity in the range from 50 mPa*s to 250 Pa*s, preferably in the range from 100 mPa*s to 100 Pa*s and more preferably in the range from 200 mPa*s to 10 Pa*s, this parameter being measured by means of rotational viscometry at 110° C. The measurement can be performed to DIN EN ISO 3219 at 30 s⁻¹, for which purpose, for example, two 20 mm plates can be used.

The acid number of the polymer with polyester units is preferably in the range from 0 to 20 mg KOH/g, more preferably in the range from 1 to 15 mg KOH/g and most preferably in the range from 6 to 10 mg KOH/g. This property can be measured by titration with NaOH (cf. DIN 53402).

Of particular interest are especially polymers with polyester units which have a hydroxyl number in the range from 0 to 200 mg KOH/g, preferably in the range from 1 to 150 mg KOH/g and most preferably in the range from 10 to 140 mg KOH/g. This property is measured to ASTM E222.

The molecular weight of the polymer with polyester units is not critical per se, although the viscosities detailed above must be observed. Depending on the structure of the polymer, it may have a relatively high molecular weight. Appropriately, the polymer may have a molecular weight in the range from 1000 g/mol to 400 000 g/mol, preferably 1500 to 100 000 g/mol and most preferably 1800 to 20 000 g/mol. This parameter refers to the weight-average molecular weight (Mw), which can be measured by means of gel permeation chromatography, the measurement being effected in DMF and polyethylene glycols being used as a reference (cf., inter alia, Burgath et. al in Macromol. Chem. Phys., 201 (2000) 782-791). In this method, a calibration curve obtained using polystyrene standards is used. This parameter therefore constitutes an apparent value.

In addition, the molecular weight of the polymers for use in accordance with the invention can be determined from the acid number and the hydroxyl number if the components are known. This process is suitable especially for polymers with a small molecular weight. For instance, preferred polymers may have a molecular weight determined from the acid number and the hydroxyl number in the range from 1000 to 30 000 g/mol, preferably 1500 to 15 000 g/mol.

The polymer with polyester units may, for example, have a linear structure. In order to observe the parameters detailed above, these polymers in many cases have a relatively low molecular weight which is preferably in the range from 1000 g/mol to 20 000 g/mol, more preferably 1500 g/mol to 5000 g/mol. Particularly suitable linear polymers with polyester units are available, inter alia, under the Dynacoll® trade name from Degussa GmbH, particular preference being given especially to Dynacoll® 7362. Dynacoll® 7362 is a polyester with a hydroxyl number in the range from 47 to 54, a molecular weight of approx. 2000 g/mol, a melting point of 53° C. and a viscosity of 0.5 Pa*s, measured at 80° C. by means of rotational viscometry. The molecular weight of Dynacoll® 7362 can be determined especially from the acid number and the hydroxyl number.

Of particular interest are encapsulation materials which, besides at least one glyceride, additionally comprise at least one hyperbranched polymer which has a hydrophilic core with polyester units and hydrophobic end groups.

It has surprisingly been found that when hyperbranched polymers are used as a constituent of the encapsulation material, many advantages can be achieved. For example, the encapsulation processes can be performed with significantly reduced amounts of solvents and/or compressed gases.

The hyperbranched polymer can thus itself function as a solvent/dispersant. The solvent/gas concentrations reduced as a result lead to safer processes compared to the prior art, since hyperbranched polymers cannot form explosive vapors like other prior art solvents.

Preferred preparations comprise a hyperbranched polymer with a hydrophilic core. “Hydrophilic” means that the core is capable of absorbing a high proportion of water. In a preferred aspect of the present invention, the hydrophilic core is soluble in water. The solubility in water at 90° C. is preferably at least 10 percent by mass, more preferably at least 20 percent by mass. This parameter is measured using the hyperbranched polymer before the hydrophobization, i.e. on the hydrophilic core as such. The measurement can be effected by the so-called flask method, which measures the water solubility of the pure substance.

In this method, the substance (solids must be pulverized) is dissolved in water at a temperature slightly above the test temperature. When saturation has been attained, the solution is cooled and kept at the test temperature. The solution is stirred until equilibrium has been attained. Alternatively, the measurement can be performed directly at the test temperature when appropriate sampling ensures that the saturation equilibrium has been attained. The concentration of the test substance in the aqueous solution, which must not comprise any undissolved substance particles, is then determined by a suitable analysis method.

The hydrophilic core preferably has a hydroxyl number measured before the hydrophobization in the range from 400 to 600 mg KOH/g, more preferably in the range from 450 to 550 mg KOH/g. This property is measured to ASTM E222. In this method, the polymer is reacted with a defined amount of acetic anhydride. Unconverted acetic anhydride is hydrolyzed with water. Subsequently, the mixture is titrated with NaOH. The hydroxyl number corresponds to the difference between a comparative sample and the value measured for the polymer. In this measurement, the number of acid groups of the polymer has to be taken into account.

In an appropriate embodiment, the hyperbranched polymer has a core which comprises polyester units. Hyperbranched polymers with polyester units are detailed especially in EP 0 630 389. In general, the hydrophilic core has a central unit which is derived from an initiator molecule having at least 2 and preferably at least 3 hydroxyl groups, and repeat units which are derived from monomers having at least one carboxyl group and at least 2 hydroxyl groups.

The terms “initiator molecule” and “repeat unit” are widely known in the technical field. It is thus possible to obtain the hyperbranched polymers by polycondensation, in which case, proceeding from a polyhydric alcohol, the carboxylic acid groups of the monomers are converted first. This forms ester groups. Since the monomers comprise at least 2 hydroxyl groups, the macromolecule after each reaction has more hydroxyl groups than before the reaction.

The initiator molecule is preferably an aliphatic polyol with preferably 3, 4, 5, 6, 7 or 8, more preferably 3, 4 or 5, hydroxyl groups.

The initiator molecule is more preferably selected from ditrimethylolpropane, ditrimethylolethane, dipenta-erythritol, pentaerythritol, alkoxylated pentaerythritol, trimethylolethane, trimethylolpropane, alkoxylated trimethylolpropane, glycerol, neopentyl alcohol, dimethylolpropane and/or 1,3-dioxane-5,5-dimethanol.

In a particular aspect of the present invention, the repeat units are derived from monomers having one carboxyl group and at least 2 hydroxyl groups. These preferred monomers include especially dimethylpropionic acid, α,α-bis(hydroxy-methyl)butyric acid, α,α,α-tris(hydroxymethyl)acetic acid, α,α-bis(hydroxymethyl)valeric acid, α,α-bis(hydroxy)-propionic acid and/or 3,5-dihydroxybenzoic acid.

The hydrophilic core is most preferably obtainable by polymerization of dimethylolpropionic acid, in which case the initiator molecule used is more preferably ditrimethylolpropane, trimethylolpropane, ethoxylated pentaerythritol, pentaerythritol or glycerol.

The hydrophilic core preferably has a molecular weight of at least 1500 g/mol, preferably at least 2500 g/mol. This parameter refers to the weight-average molecular weight (Mw), which can be measured by means of gel permeation chromatography, the measurement being effected in DMF and polyethylene glycols being used as a reference (cf., inter alia, Burgath et al. in Macromol. Chem. Phys., 201 (2000) 782-791). In this case, a calibration curve which has been obtained using polystyrene standards is used. This parameter therefore constitutes an apparent value.

The hydrophilic core may preferably have a glass transition temperature which is in the range from −40 to 60° C., more preferably 0 to 50° C. and most preferably 10 to 45° C. The glass transition temperature can be determined by the DSC method, in which a heating rate of 3° C./min can be used (DMA tan δ peak; Netsch DMA 242 3-point bending 1 Hz 3° C./min).

The hydrophobization of the surface of the polymer is generally obtained as the last reaction step by reacting at least some of the free hydroxyl groups with preferably a long-chain carboxylic acid.

The degree of functionalization of the hyperbranched core molecule with hydrophobic end groups, preferably with fatty acid-containing units, is preferably at least 5%, especially preferably at least 30%, more preferably at least 40%. In a further aspect of the present invention, the degree of functionalization of the hyperbranched core molecule with hydrophobic end groups, preferably with fatty acid-containing units, is in the range from 30 to 100%, preferably in the range from 35 to 95%, especially preferably in the range from 40 to 90% and most preferably in the range from 45 to 85%.

The degree of functionalization is based on the proportion of hydroxyl groups which are converted in the hydrophobization. Accordingly, the degree of functionalization or the degree of esterification with fatty acids can be determined via the measurement of the hydroxyl number for the hyperbranched core molecule before the hydrophobization reaction and after the hydrophobization reaction.

In addition to the hydrophilic core, the hyperbranched polymer has hydrophobic end groups. In this connection, the term “hydrophobic end groups” means that at least some of the chain ends of the hyperbranched polymer have hydrophobic groups. In this context, it can be assumed that an at least partly hydrophobized surface is obtained as a result.

The term “hydrophobic” is known per se in the technical field, and the groups which are present at least on some of the ends of the hyperbranched polymers, considered per se, have a low water solubility.

In a particular aspect, the surface is hydrophobized by groups which are derived from carboxylic acids having at least 6, preferably at least 12 carbon atoms. The carboxylic acids preferably have at most 40, particularly at most 32 carbon atoms, more preferably at most 20 carbon atoms and most preferably at most 18 carbon atoms. The groups may be derived from saturated and/or unsaturated fatty acids. The proportion of the carboxylic acids having 12 to 18 carbon atoms is preferably at least 30% by weight, more preferably at least 50% by weight and most preferably at least 60% by weight, based on the weight of the carboxylic acids used for the hydrophobization.

These include especially fatty acids which are present in linseeds, soybeans and/or tall oil. Particularly suitable fatty acids are those which have a low proportion of double bonds, for example hexadecenoic acid, especially palmitoleic acid, and octadecenoic acid, especially oleic acid.

Preferred carboxylic acids in this context have a melting point of at least 35° C., preferably at least 40° C. and more preferably at least 60° C. Accordingly, preference is given to using linear, saturated carboxylic acids. These include especially octanoic acid, decanoic acid, dodecanoic acid, tetradecanoic acid, hexadecanoic acid, heptadecanoic acid, octadecanoic acid, eicosanoic acid, docosanoic acid and tetracosanoic acid. Particular preference is given to saturated fatty acids having 16 to 22 carbon atoms, especially preferably 16 to 18 carbon atoms.

Of particular interest are especially hyperbranched polymers (after the hydrophobization) which have a molecular weight of at least 3000 g/mol, preferably at least 6000 g/mol, more preferably at least 7500 g/mol. The molecular weight is preferably at most 30 000 g/mol, more preferably at most 25 000 g/mol. This parameter refers to the weight-average molecular weight (Mw), which can be measured by means of gel permeation chromatography, the measurement being effected in DMF and the reference used being polyethylene glycols (cf., inter alia, Burgath et al. in Macromol. Chem. Phys., 201 (2000) 782-791). In this method, a calibration curve which has been obtained using polystyrene standards is used. This parameter is therefore an apparent value.

The polydispersity Mw/Mn of preferred hyperbranched polymers is preferably in the range from 1.01 to 6.0, more preferably in the range from 1.10 to 5.0 and most preferably in the range from 1.2 to 3.0, where the number-average molecular weight (Mn) can likewise be obtained by GPC.

The weight ratio of hydrophilic core to the hydrophobic end groups may preferably be in the range from 10:1 to 1:10, more preferably from 1:1 to 1:2.5. This ratio arises from the weight average of the hydrophilic core and the weight average of the hyperbranched polymer.

The degree of branching of the hyperbranched polymer is in the range from 20 to 70%, preferably 25 to 60%. The degree of branching depends on the components used to prepare the polymer, especially the hydrophilic core, and the reaction conditions. The degree of branching can be determined according to Frey et al., this process being detailed in D. Hölter, A. Burgath, H. Frey, Acta Polymer, 1997, 48, 30 and H. Magnusson, E. Malmstrom, A. Hult, M. Joansson, Polymer 2002, 43, 301.

The hyperbranched polymer preferably has a melting temperature of at least 30° C., more preferably at least 35° C. and most preferably at least 40° C. In a particular aspect of the present invention, the melting point of the hyperbranched polymer may preferably be at most 65° C., especially preferably at most 60° C., more preferably at most 57° C. and most preferably at most 55° C. The melting temperature can be determined by means of differential scanning calorimetry (DSC), for example with the Mettler DSC 27 HP apparatus and a heating rate of 10° C./min.

The water solubility of the hyperbranched polymer after the hydrophobization is preferably at most 10% by mass, more preferably at most 7% by mass and most preferably at most 5% by mass, measured by the flask method detailed above at 40° C.

The hyperbranched polymer preferably consists essentially of hydrogen, oxygen and carbon. The term “essentially” means that further elements are present in the hyperbranched polymer up to at most 10% by weight, more preferably at most 5% by weight.

In a particular aspect of the present invention, the hyperbranched polymer can be degraded enzymatically. This can be achieved, for example, by virtue of the hydrophilic core and/or the hydrophobic shell comprising enzymatically degradable organic ester groups.

The preparation of these hyperbranched polymers is detailed especially in EP 630 389. In general an initiator molecule can be reacted with at least one compound which comprises at least two hydroxyl groups and at least one carboxylic acid group. Thereby a hydrophilic core is obtained which can be reacted with at least one hydrophobic compound, for example a long-chain carboxylic acid.

In general, the reaction is carried out at a temperature in the range from 0° C. to 300° C., preferably from 100° C. to 250° C., and the reaction can be accelerated by known esterification catalysts. These catalysts include, for example, Lewis and Brønsted acids, especially p-toluenesulfonic acid, methanesulfonic acid, trifluoroacetic acid, BF₃, AlCl₃ and SnCl₄; titanium compounds, especially tetrabutyl titanate; zinc powder and/or tin powder.

Preferably water released in the esterification is removed from the reaction mixture.

The proportion of polymer with polyester units, based on the weight of the preparation, is preferably in the range from 1 to 98.5% by weight, more preferably in the range from 10 to 90% by weight and most preferably in the range from 20 to 80% by weight.

The weight ratio of polymer with polyester units to glyceride with a melting point of at least 35° C. is not critical per se. Surprisingly, however, the proportion of natural substance can be increased by a high proportion of polymer, such that the degree of loading can surprisingly be enhanced by this measure. On the other hand, the storage stability and the enzymatic degradability can be improved by the use of a high proportion of glyceride, this finding being surprising. Of particular interest are therefore preparations which feature a weight ratio of polymer with polyester units to glyceride which is preferably in the range from 20:1 to 1:20, more preferably 10:1 to 1:10 and most preferably 5:1 to 1:5.

In addition to an encapsulation material, the inventive preparations comprise at least one bioactive natural substance. The bioactive natural substance is preferably bound to the encapsulation material by a noncovalent method. This can be done, for example, by ionic or polar interactions or by van der Waals forces.

Because of the interaction of encapsulation material and bioactive natural substance, the preparation of the present invention can differ from a conventional mixture of these components.

This interaction can be measured in a known manner. Depending on the natural substance, spectroscopic methods are suitable for this purpose in many cases. For example, it is possible in some cases to observe shifts in the infrared spectrum.

In addition, the inventive preparations, compared to a conventional mixture, can exhibit delayed release of the bioactive natural substance into a medium other than the bioactive natural substance of the preparation. The delayed release can be measured according to the method described by Smirnova, I.; Suttiruengwong, S.; Arlt, W. “Feasibility study of hydrophilic and hydrophobic silica aerogels as drug delivery systems”; Journal of Non-Crystalline Solids (2004) 54-60.

In general, the time difference in order to obtain an identical concentration of the bioactive natural substance in the medium into which the natural substance is released is at least 1 minute, preferably at least 5 minutes. In this context, this time difference is based on the measurement of a preparation of the present invention and the measurement of a conventional mixture of these components under identical delayed release conditions. Delayed release means that the conditions are not selected such that the preparation releases the bioactive natural substance as fast as possible. These conditions are familiar to those skilled in the art with knowledge of this application. The values of the conventional mixture can also be determined by separate addition of the components.

In order to achieve controlled release, the preparation is preferably present in encapsulated form, the term “encapsulation” being known in the technical field. In one aspect, the bioactive natural substance can be embedded, for example, in a shell which comprises the encapsulation material. In a preferred embodiment, it is also possible that a matrix encapsulation exists, in which case the encapsulation material protects the bioactive natural substance in a matrix. The bioactive natural substance is preferably dispersed homogeneously in the encapsulation material. The matrix more preferably forms, together with the bioactive natural substance, a homogeneous phase, such that the bioactive natural substance is present dissolved in the encapsulation material. According to these statements, the bioactive natural substance can be protected by a matrix encapsulation and/or a core-shell encapsulation. The inventive preparations may appropriately be present in the form of microcapsules or microparticles.

According to the invention, the term “microcapsules” is understood to mean particles or aggregates which comprise an inner space or core which is filled with a solid, gelated, liquid or gaseous medium and is surrounded by a continuous shell of encapsulation material. These particles preferably have small dimensions.

In addition, the microscopically small capsules may comprise one or more cores distributed in the continuous encapsulation material consisting of one or more layers. The distribution of the material to be enveloped may be to such an extent as to give rise to a homogeneous mixture of shell and core material, which is referred to as a matrix. Matrix systems are also known as microparticles.

According to the statements made above, the preparation of the present invention may be in particulate form. In this case, these particles preferably have a size in the range from 1 to 1000 μm, more preferably 10 to 500 μm.

The form of the particles is uncritical per se, but the particles preferably have a spherical form.

In the context of the present invention, the term “spherical” means that the particles preferably have a spherical shape, though it is obvious to the person skilled in the art that, owing to the preparation methods, it is also possible for particles of another shape to be present, or that the form of the particles can deviate from the ideal sphere shape.

Accordingly, the term “spherical” means that the ratio of the longest dimension of the particles to the shortest dimension is not more than 4, preferably not more than 2, these dimensions each being measured through the center of the particles. Preferably at least 70%, more preferably at least 90%, based on the number of particles, are spherical.

The particle size can be determined in a commonly known manner. For this purpose, it is possible, for example, to use microscope images which can be evaluated visually and/or with the aid of computers.

In addition, preferred microparticles have a particularly narrow particle size distribution. Preferably at least 80% by weight of the particles are thus within a size range from 1 μm to 200 μm, preferably 1 μm to 100 μm, more preferably 1 μm to 50 μm.

In a particular aspect of the present invention, preferably 90% of the particles have a size in the range from 1 to 1000 μm, especially preferably 3 to 800 μm, more preferably 7 to 700 μm and most preferably 10 to 500 μm.

Preferred preparations according to the present invention exhibit a readily controllable enzymatic degradability. Of particular interest are preparations which can be degraded within three days or less, preferably 2 days or less and more preferably one day or less. Preferably at least 20% by weight, more preferably at least 50% by weight, of the natural substance present in the preparation is released on contact with an enzyme within at most three days, preferably within at most two days and more preferably within 24 hours. In this context, preparations are degraded with a suitable enzyme, especially a lipase, for example Lipomod® 34P (Biocatalyst Lmt., UK). For example, preparations with a degree of loading of 1 to 20% by weight can be examined, in which case preferably 1% by weight of active ingredient-laden polymer particles can be suspended in 50 ml of phosphate buffer (pH 5.01) or in 50 ml of solution of the enzyme Lipomod® 34P (Biocatalyst Lmt., UK) in the same buffer (with a concentration of Lipomod® 34P of 0.5 mg/ml). The samples can be kept in a water bath at 37° C. without mixing. At regular time intervals, for example 5 hours, samples of approx. 5 ml can be taken, and the concentration of the active ingredient can be analyzed by suitable methods, for example iodometric titration with a Metrohm Titroprocessor 686. In this context, the release of a comparative sample which does not comprise an enzyme is taken into account in order to rule out problems of storage stability under the test conditions selected, such that the value reported above is calculated from the difference between measured sample and comparative sample. The enzymatic degradability of the preparation arises especially through the degradability of the encapsulation material. The polymers with polyester groups and the glycerides are preferably enzymatically degradable. The enzymatic degradation of the glyceride can be effected analogously to the method detailed above, except that the concentration increase of the fatty acid is determined. The situation is similar for the degradation of the polymer, it being possible here to measure the increase in concentration of monomers suitable for forming polyesters.

The preparations of the present invention may exhibit an outstanding shear stability which can in many cases be influenced via the selection of the encapsulation material and the process conditions in the preparation production. The preparations preferably exhibit a shear stability of 1 minute or longer, preferably 5 minutes or longer, this stability being determined at a load which corresponds to that of an ULTRA-TURRAX® stirrer at 15 000 revolutions per minute, preferably 20 000 revolutions per minute. In general, a dispersion of the particles, for example in a pharmaceutical oil (WINOG 100 Pharma paraffin oil from Univar GmbH), is prepared here, and the dispersion may contain, for example, 10% by weight of preparation. Before and after the stability measurement, which can be effected, for example, by means of an ULTRA-TURRAX® stirrer at 15 000 revolutions per minute, preferably 20 000 revolutions per minute, the particles are analyzed microscopically to assess the particle form, size and distribution. There is shear stability under the aforementioned conditions if no significant changes can be observed.

The storage stability of the inventive preparations is in many cases likewise surprisingly high, and depends on the type and composition of the medium if the preparations are stored in the form of dispersions or emulsions, and/or the storage temperatures. Under preferred storage conditions, preferred preparations can be stored over a long period, for example 10 days or longer, preferably 30 days or longer and more preferably 90 days or longer. This parameter can be measured by the release of active ingredient into a medium or the degradation of the bioactive natural substance. These values refer to the period up to which at most 10% of the bioactive natural substance has been released into a medium in which the preparation can be stored, or the time up to which at most 10% of the bioactive natural substance has been degraded, for example by oxidation.

The term “bioactive natural substance” encompasses substances which have an effect on biological systems and can be obtained from natural sources, especially from plants or animals.

The bioactive natural substance preferably has a molar mass in the range from 50 g/mol to 100 000 g/mol, more preferably 100 g/mol to 5000 g/mol and most preferably 160 g/mol to 1500 g/mol.

The bioactive natural substance can be selected from a wide area, particular preference being given especially to natural substance extracts, especially phytoextracts. Natural substance extracts are substances or substance mixtures which can be obtained by extraction of natural substances, whereby phytoextracts are obtained from plants. Natural substance extracts are typically not subjected to a chemical conversion but are merely obtained from the natural substances by physical methods, amongst others extraction, distillation and precipitation methods. Such substances are therefore very sought-after in the cosmetic and pharmaceutical industries owing to high consumer acceptance.

The preferred natural substance extracts include compositions obtained by extraction from fruit constituents (pits, peel, juices), for example of pineapple, apple, banana, pear, strawberry, grapefruit, peach, apricot, pomegranate, lingonberry, cranberry, cherry, raspberry, blackcurrant, coffee, mango, orange, passion fruit, sour cherry, grape, quince, soy bean, olive, cocoa, nut or sloe, but also from plant constituents (leaves, wood, roots), for example from vanilla, chamomile, coffee, tea, oak, olibanum or spices, or from products of the foods industry, for example rum, beer, cognac, tequila, brandy, whisky, coffee oil and malt. These extracts can in many cases be obtained commercially. These include especially Cocoa Absolute 14620, Cocoa LC 10167, Cocoa P 11197, Cocoa U88; all available from Degussa GmbH. Natural extracts in this context are extracts which can be obtained from natural sources or which have properties similar to these extracts.

The preferred bioactive natural substances additionally include especially flavorings, aromas, natural extracts, enzyme-modified food additives, natural waxes, proteins, peptides, vitamins and vitamin precursors, fats and fatty acids, amino acids and amino acid precursors, for example creatin, sugar and sugar derivatives, nucleotides, nucleic acids and precursors and derivatives thereof, for example DNA and RNA oligomers, medicaments, enzymes and coenzymes.

Of particular interest are, among other substances, bioactive natural substances which comprise at least one compound selected from the group consisting of tocopherol and derivatives, ascorbic acid and derivatives, deoxyribonucleic acid, retinol and derivatives, alpha-lipoic acid, niacinamide, ubiquinone, bisabolol, allantoin, phytantriol, panthenol, AHA acids, amino acids, hyaluronic acid, polyglutamic acid, beta-glucans, creatin and creatin derivatives, guanidine and guanidine derivatives, ceramides, sphingolipids, phytosphingosine and phytosphingosine derivatives, sphingosine and sphingosine derivatives, sphinganine and sphinganine derivatives, pseudo-ceramides, essential oils, peptides, proteins, protein hydrolysates, plant extracts and vitamin complexes. Derivatives of the substances detailed above are especially compounds which have an essentially similar action to the particular substance per se. In many cases, organisms can convert the derivatives to the corresponding substance, such that these derivatives achieve an effect similar to the administration of the particular substance.

The preferred coenzymes include especially coenzyme Q10 (2,3-dimethoxy-5-methyl-6-polyprenyl-1-benzoquinone).

The vitamins include especially vitamin A, vitamins of the B complex, for example vitamin B1, vitamin B2, vitamin B3 (folic acid) and vitamin B12, vitamin C (ascorbic acid), vitamins of the D complex, especially 7-dehydrocholesterol, lumisterol, calciferol, ergocalciferol, cholecalciferol, 22,23-dihydroergocalciferol and sitocalciferol, and vitamin E (tocopherol) and vitamin K (phylloquinone, menaquinone).

The preferred amino acids include especially DL-methionine, L-lysine, L-threonine, L-tryptophan, L-theanine and L-leucine.

The aromas include especially alkanes, alkenes, ketones, aldehydes, sulfur compounds, heterocycles, carboxylic esters, alcohols and/or natural extracts, for example limonene or linalool.

Examples of fats are especially oils which derive from vegetable or animal material, and which can be used in accordance with the invention, are olive oil, palm oil, rapeseed oil, flax oil, and oils of the seeds or pips of, for example, sunflower, apple, pear, citrus fruits such as mandarin, orange, grapefruit or lemon, melon, pumpkin, raspberry, blackberry, elder, blackcurrant, pomegranate, wheat and rice germs, and cottonseeds, and soya and palm kernels. Oils derived from animal tallow are especially bovine tallow, bone oil, fish oils, preference being given especially to PUFA-containing oils (polyunsaturated fatty acids) which have a high proportion of omega-3 fatty acids and omega-6 fatty acids.

Particularly preferred oils are especially kernel oils. Kernel oils can be extracted especially from the pips or seeds of plants, for which especially residues from the processing of fruits and berries, and more preferably from juice production, are suitable. Especially residues from the processing of, for example, apple, peach, pear, citrus fruits such as mandarin, orange, grapefruit or lemon, melon, pumpkin, raspberry, blackberry, elder, cherry, rosehip, apricot, strawberry, blackcurrant and pomegranate are useful as starting materials. Processes for obtaining preferred oils from vegetable constituents by extraction are known especially from publication DE A 10 2005 037 209. Kernel oils in many cases have a high content of triglycerides of unsaturated fatty acids. For instance, the proportion of oleic acid and/or linoleic acid is in many cases 50 to 90% by weight. In addition, kernel oils are rich in vitamins, for example vitamin A, and/or minerals.

In addition, it is especially possible to use carotenoids, flavonoids such as resveratrol and xanthohumol, isoflavonoids, terpenes, phytosterols such as beta-sitosterol, glycoconjugates such as aloeresin, aloenin, triterpenoids, for example 11-keto-β-boswellic acid or acetyl-11-keto-β-boswellic acid, which can be obtained from olibanum, polyphenols, especially lupeol, squalene, hydroxytyrosol, tocopherols and vanillin as the bioactive natural substance.

In addition, natural waxes are examples of preferably usable natural substances. These waxes are usually of vegetable or animal origin, which means that they are typical natural products. A strict delimitation of the vegetable and animal waxes on the basis of the fatty acids and fatty alcohols involved in their structure cannot be undertaken. However, it is undisputed that montanic acid, palmitic acid and stearic acid are typical fatty acids involved in these natural waxes. On the part of the alcohols, mention should be made here in particular of cetyl alcohol and ceryl alcohol. True natural waxes of vegetable origin are, for example, palm leaf waxes such as carnauba wax, palm wax, raffia wax, ouricury wax, grass waxes, for example candelilla wax, esparto wax, fiber wax and sugarcane wax; berry and fruit waxes are, for example, apple wax, pear wax, quince wax, japan wax, bayberry wax and myrtle wax. The best known example of a true natural wax of animal origin is beeswax, which consists principally of miricyl palmitate, i.e. of palmitic acid esterified with miricyl alcohol. Also known are Chinese insect waxes, shellac wax and wool waxes, as can be obtained, for example, from sheep's wool. Processes for obtaining preferred waxes from vegetable constituents by extraction are known especially from publication DE A 10 2005 037 210.

The bioactive natural substances detailed above can be used individually or as a mixture of two, three or more. In this case, the mixtures may comprise natural substances of the same class or of different classes. For example, a combination may comprise a mixture which has one or more fruit waxes and/or one or more kernel oils.

The inventive preparations may have a surprisingly high proportion of bioactive natural substances. In a particular aspect of the present invention, the weight ratio of encapsulation material to natural substance may preferably be in the range from 40:1 to 0.5:1, more preferably in the range from 20:1 to 2:1. The degree of loading may preferably be within a range from 1% to 95%, more preferably 5% to 90%, further preferably 10 to 60% and very preferably 10 to 30%, the degree of loading being given by the proportion by weight of the bioactive natural substance in the total weight of the preparation.

The bioactive natural substance can in principle be released from the inventive preparation in any desired manner. For example, an enzymatic degradation can be effected in order to release the substance to be released. In this case, the release period can be controlled by the degradation rate.

In addition, the release can be controlled via a change in the pH value, temperature, pH, radiation frequency and type of medium.

The type of medium can be altered, for example, via the addition of solvents, surfactants or salts. The solvents used to vary the medium may include water or alcohols, such as ethanol or isopropanol.

In addition, the release rate can be controlled by the proportion of polymer with polyester units. In this context, the degree of functionalization of the hyperbranched polymer or the hydroxyl number of the polymer are parameters which can be used to control the release. According to the degree of functionalization of the hyperbranched polymer and the medium into which the substance to be released is to be released, a wide variety of different solvents can thus be added in order to achieve a very retarded or a very rapid release. If the encapsulated natural substance is to be released in polar media, the more OH groups of the hyperbranched core polyester have been esterified/functionalized with fatty acids, the slower the release is. This effect can be promoted by adding appropriate solvents.

In addition, the active ingredient release can be controlled especially via the proportion of encapsulation material which protects the bioactive natural substance, and/or the degree of functionalization/degree of hydrophobization or the hydroxyl number of the polymer with polyester units.

The higher the proportion of encapsulation material in the preparation, the greater the release period is. It has been found that, surprisingly, when hyperbranched polymers are used, the concentration in the starting mixture can also be increased above the polymer concentrations of 10 percent by mass which are customary in the prior art up to a polymer concentration of 70 percent by mass. The ultimately selected concentration of glyceride and of polymer, together with the temperature regime or the alteration of pH or dissolution power of the solvent, decides the proportion of encapsulation material and hence the release period.

In order to produce the inventive preparations, the low molecular weight compounds and the encapsulation material can be combined with one another. For this purpose, various methods, especially RESS, GAS, PCA, SEDS and/or PGSS methods, are suitable. Such methods are widely known per se and are described, for example, in Gamse et al., Chemie Ingenieur Technik 77 (2005), No. 6, pages 669 to 679.

Of particular interest are especially processes for producing the inventive preparations, comprising the steps of
a) producing a melt comprising at least one polymer with polyester units, at least one glyceride with a melting point of at least 35° C. and at least one bioactive natural substance,
b) introducing the melt into a second liquid phase in which the encapsulation material is sparingly soluble, said second liquid phase having a solidification temperature below the solidification temperature of the encapsulation material,
c) dispersing the melt in the second liquid phase at a temperature which is greater than or equal to the solidification temperature of the encapsulation material and
d) solidifying the melt dispersed in the second liquid phase.

The above-detailed process for producing the inventive preparations can be performed in a particularly simple and economically viable manner, it being possible especially to dispense with the use of solvents harmful to health. In addition, a series of further advantages can be achieved. One of these is that the above-detailed process succeeds in the production of preparations, especially of microparticles, without a high level of apparatus complexity and without the use of solvents harmful to health. The process according to the invention can especially provide particularly homogeneous microparticles with a given particle size and particle size distribution. In this context, the process is particularly flexible. For instance, it is possible to produce either small or large particles with a relatively narrow particle size distribution in each case with one plant. Moreover, it is possible to form microparticles which comprise pulverulent and/or liquid natural substances. In addition, the bioactive natural substances may also be soluble in the encapsulation material or be a solvent for the encapsulation material. Furthermore, the process can be performed at relatively low temperatures, such that thermally sensitive natural substances can be encapsulated. Furthermore, the process of the present invention can be performed with a high throughput, such that large amounts of particles can be formed within a short time. The present process can form the particles continuously. The plants for performing the process according to the invention generally require only very low capital and operating costs, since the plants can also be operated at standard pressure and in many cases no explosive mixtures are formed, generally making it possible to do without the use of substances harmful to health. In the course of operation, the plants generally require only small amounts of energy. Furthermore, the plants in many cases have low complexity, such that the maintenance costs are low and the plants can be controlled in a simple and reliable manner.

In a preferred process for producing the inventive preparations, a melt comprising at least one polymer with polyester units, at least one glyceride with a melting point of at least 35° C. and at least one bioactive natural substance is produced. The bioactive natural substance is preferably distributed finely in the melt which comprises the glyceride and the polymer with polyester units. For this purpose, it is possible to use known apparatus, for example stirrers, which include a stirred tank with a propeller stirrer, disk stirrer, toothed disk stirrer, anchor stirrer, helical stirrer, blade stirrer, paddle stirrer, pitched-blade stirrer, cross-blade stirrer, spiral stirrer, MIG® stirrer, INTERMIG® stirrer, ULTRA-TURRAX®, screw stirrer, belt stirrer, finger stirrer, basket stirrer, impeller stirrer, as well as dispersers and homogenizers which can work, inter alia, with ultrasound. The apparatus may generally have at least one shaft on which in turn preferably 1 to 5 stirrer elements may be mounted.

This may give rise, for example, to a solution, a suspension or a dispersion, the particle size of the phase present in distributed form being preferably at most 5000 μm, more preferably at most 1000 μm, if the bioactive natural substance is present in particulate form.

The parameters necessary for this purpose depend on the apparatus detailed above. The stirrer speed may preferably be in the range from 10 to 25 000 revolutions per minute, more preferably in the range from 20 to 20 000 revolutions per minute.

The temperature at which the melt is produced may likewise be within a wide range, which depends inter alia on the solidification temperature of the polymer with polyester units or the glyceride. The temperature is preferably in the range from 40° C. to 200° C., more preferably in the range from 45° C. to 100° C. The pressure used in the production of the melt is likewise uncritical, and depends in many cases on the type of bioactive ingredient and the solidification temperature of the encapsulation material. For example, the pressure may be selected within the range from 0.1 mbar to 200 bar, preferably in the range from 10 mbar to 100 bar.

In a particular aspect of the present invention, preferably no solvent, especially no organic solvent, is added to the melt, particularly preferred melts not comprising any solvent. A solvent is understood here to mean a substance in which the encapsulation material is soluble and which has to be removed during the production process, since this compound should not be present in the preparations, especially the microparticles. In this context, it has to be noted that many of the natural substances detailed above may have properties of a solvent. However, these substances are an intended constituent of the microcapsules, and so these compounds are not solvents in the context of the present invention. Accordingly, the use of solvents to perform the process is not necessary. On the other hand, some of the natural substances are supplied in dissolved form, in which case the solvents used for this purpose are generally uncritical for the use of the natural substance, such that they are, for example, not harmful to health. Such auxiliaries need not necessarily be removed before the production of the melt. Instead, these auxiliary substances may be incorporated into the melt.

The melt described above is, in accordance with the invention, transferred to a second liquid phase in which the encapsulation material is sparingly soluble and which has a solidification temperature below the solidification temperature of the encapsulation material. Accordingly, the second liquid phase comprises one or more substances which are immiscible with the encapsulation material and which serve as the main constituent of the continuous phase. Since the encapsulation material or the melt is hydrophobic, the second liquid phase is accordingly preferably hydrophilic.

The term “sparingly soluble” means that the solubility of the encapsulation material in the second liquid phase should be at a minimum. The solubility depends in many cases on the temperature. Accordingly, the dispersing conditions may in many cases be selected such that a minimum proportion of the encapsulation material is dissolved in the second liquid phase. The encapsulation material, especially the polymer with polyester units and the glyceride, preferably has a solubility by the flask method at the dispersing temperature of at most 20 percent by mass, preferably at most 10 percent by mass, in the second liquid phase. In a particular aspect, the encapsulation material may preferably have a solubility by the flask method at 40° C. of at most 20 percent by mass in the second liquid phase.

The second liquid phase has a solidification temperature below the solidification temperature of the encapsulation material. In general, this temperature arises from the melting temperature or the glass transition temperature of the main constituent of the second liquid phase, and freezing point depressions may occur as a result of auxiliaries or additives or as a result of the use of substance mixtures. This parameter can be obtained from DSC measurements, the melting points or freezing points of the customary main constituents of the second liquid phase being listed in reference works.

The hydrophilic substances which may be present as the main constituent in the second liquid phase include especially water and alcohols having 1 to 7, preferably 1 to 4, carbon atoms, especially methanol, ethanol, propanol and/or butanol, particular preference being given to water.

The second liquid phase may comprise besides the main constituent additional auxiliaries, especially dispersants and stabilizers. These auxiliaries are known in the technical field, and dispersants counteract aggregation of the particles. These include especially emulsifiers, protective colloids and surfactants, each of which may be selected according to the utilised encapsulation materials, bioactive natural substances and the main constituent of the second liquid phase. The preferred surfactants include especially anionic surfactants such as lauryl ether sulfate, cationic surfactants and nonionic surfactants, for example polyvinyl alcohols and ethoxylated fatty alcohols. Stabilizers may be used for a multitude of uses, and these auxiliaries maintain or stabilize a desired unstable state. These include especially antisettling agents such as pectins and/or carrageenan.

The second liquid phase preferably comprises 60 to 100% by weight of main constituent, for example the hydrophilic substances detailed above, such as water or alcohols having up to 4 carbon atoms. In addition, the second liquid phase may contain 0 to 40% by weight of auxiliary substances, especially 0 to 20% by weight of emulsifiers and 0 to 20% by weight of stabilizers.

The melt introduced into the second liquid phase is dispersed at a temperature which is greater than or equal to the solidification temperature of the encapsulation material.

In the context of the present invention, the solidification temperature of the encapsulation material refers to the temperature at which the encapsulation material becomes solid, such that particles at this temperature no longer agglomerate to larger aggregates without external actions. Depending on the structure and crystallization properties, the solidification temperature may result, for example, from the glass transition temperature or the melting temperature of the polymer with polyester units or of the glyceride, which can be determined, for example, by DSC methods (Differential Scanning Calorimetry; Dynamic Difference Calorimetry). In this context, it has to be noted that amorphous polymers generally have only a glass transition temperature, whereas crystalline polymers exhibit a melting temperature. Partly crystalline polymers may exhibit both a glass transition temperature and a melting temperature, in which case the temperature at which the particles exhibit no agglomeration is crucial. If the surface is essentially crystalline, the melting point of these constituents is crucial.

Dispersing means in this context that the melt comprising at least one bioactive natural substance is distributed finely in the continuous second liquid phase. The dispersing can be performed here with known equipment and apparatus, for example stirrers which comprise a stirred tank with a propeller stirrer, disk stirrer, toothed disk stirrer, anchor stirrer, helical stirrer, blade stirrer, paddle stirrer, pitched-blade stirrer, cross-blade stirrer, spiral stirrer, MIG® stirrer, INTERMIG® stirrer, ULTRA-TURRAX®, screw stirrer, belt stirrer, finger stirrer, basket stirrer, impeller stirrer, as well as dispersers and homogenizers which can work, inter alia, with ultrasound. The apparatus may generally have at least one shaft on which in turn preferably 1 to 5 stirrer elements may be mounted.

The duration and the energy input of the dispersing step are dependent here on the desired particle size and particle size distribution. Accordingly, the duration of the dispersing step can be selected within a wide range. The dispersing step is performed preferably for a duration in the range from 1 second to 5 hours, more preferably in the range from 10 seconds to 2 hours.

In a particular aspect of the present invention, the Newton number in the dispersing step may preferably be in the range from 0.1 to 1000, more preferably in the range from 0.4 to 800.

The Newton number is calculated from the formula

N_{Po}=P·ρ⁻¹·n⁻³·d⁻⁵

where
P is the stirrer output [W] or [kg·m⁻²·s⁻³],
d is the diameter of the stirrer [m],
ρ is the density of the liquid in the system [kg·m⁻³] and
n is the frequency or the rotational speed [s⁻¹].

According to a particular embodiment of the process according to the invention, the Reynolds number in the dispersing step may preferably be in the range from 1 to 10⁷, more preferably in the range from 10 to 10⁶.

The Reynolds number is calculated for a stirred reactor by the following formula:

N_{Re}=n·L²·ρ·μ⁻¹

where
n is the frequency or the rotational speed [s⁻¹],
L is the characteristic length of the system [m],
ρ is the density of the liquid in the system [kg·m⁻³] and
μ is the dynamic viscosity of the liquid in the system [kg·m⁻¹s⁻¹].
The parameters needed for this purpose depend on the apparatus detailed above. The stirrer speed may preferably be in the range from 10 to 25 000 revolutions per minute, more preferably in the range from 20 to 10 000 revolutions per minute.

At the same time, the Newton number used and the stirrer speed depend on the desired particle size and particle size distribution. The more energy is supplied and the longer dispersing is continued, the smaller the particle sizes which can be achieved. A narrow particle size distribution can likewise be achieved by means of a high dispersing energy and a long dispersing time. On the other hand, long dispersing times and high dispersing energies are frequently associated with additional costs.

The temperature at which the melt is dispersed in the second liquid phase may likewise be within a wide range, which depends inter alia on the solidification temperature of the encapsulation material. The temperature is preferably in the range from 40° C. to 200° C., more preferably in the range from 45 to 100° C. The pressure used in the dispersing of the melt is likewise uncritical, and in many cases depends on the type of bioactive natural substance and the solidification temperature of the encapsulation material. For example, the pressure may be selected within the range from 10 mbar to 200 bar, preferably in the range from 100 mbar to 100 bar.

The temperature in the dispersing step is greater than or equal to the solidification temperature of the encapsulation material. The dispersing temperature is preferably 1° C. to 100° C., more preferably 5° C. to 70° C. and most preferably 10 to 50° C. above the solidification temperature of the encapsulation material.

The weight ratio of melt to the second liquid phase may be within a wide range. This ratio is preferably in the range from 1:1 to 1:200, more preferably 1:1.5 to 1:10.

In the dispersing step, the composition may comprise, for example, 50 to 99% by weight, preferably 70 to 98% by weight, of second liquid phase and 1 to 50% by weight, preferably 2 to 30% by weight, of melt.

Once the melt is present dispersed in the second liquid phase, the dispersed melt is solidified. The solidification can be effected by known methods, for example by adding salts at a temperature slightly above the solidification temperature or by cooling. Preference is given to solidifying the melt by cooling the second liquid phase to a temperature below the solidification temperature of the encapsulation material.

The type of cooling depends inter alia on the desired particle size and particle size distribution. Rapid cooling can lead, inter alia, to a particularly uniform particle size distribution and small particles, since aggregation can be prevented. At the same time, the formation of agglomerates is lower for a large cooling volume.

In addition, the particle size distribution and the size of the particles can be influenced by means of auxiliaries, for example dispersants and emulsifiers. These additives may be added, for example, to the second phase, in which case additization of the surface of the particles formed can be achieved. This additization can also prevent aggregation of the microparticles during drying or in the course of storage.

Depending on the application, the composition thus obtained can be processed further directly without undertaking a purification, concentration or separation. In a particular embodiment, the present process may comprise the step of separating the microparticles formed in the second liquid phase. The separation can be effected by known processes, especially by filtration, centrifugation, sedimentation, magnet separation, flotation, sieving or decanting, and the processes may be used individually or in combination. This can essentially completely remove the compounds of the second liquid phase, such that dried microparticles are obtained, or the particles can be concentrated.

The apparatus usable to separate or concentrate the microparticles, also referred to hereinafter as separators, are common knowledge. For instance, it is possible to use apparatus including centrifuges, decanters, centrifugal separators, filters, for example gravity filters, suction filters (vacuum filters), pressure filters, suction/pressure filters, press filters, vacuum drum filters, belt filters, disk filters, planar filters, chamber filter presses, frame filter presses, candle filters, leaf filters, membrane filter plates and/or filter belt presses.

The temperature in the separation or concentration step may likewise be within a wide range, which depends upon factors including the solidification temperature of the encapsulation material. In order to prevent aggregation of the particles, the selected temperature should be below the solidification temperature of the encapsulation material. The temperature is preferably in the range from −20° C. to 80° C., more preferably in the range from −10° C. to 40° C. The pressure used in the separation or concentration is likewise uncritical, and depends in many cases on the type of bioactive natural substance and the solidification temperature of the encapsulation material. For example, the pressure may be selected within the range from 10 mbar to 200 bar, preferably in the range from 100 mbar to 100 bar.

After the separation step, the resulting particles can be washed. To this end, the particles can be treated with a wash liquid in order to separate from the particles additive residues and/or bioactive natural substances present on the surface of the particles. Accordingly, the particles, especially the encapsulation material, should not be soluble in the wash liquid. On the other hand, the substance to be removed, for example the bioactive natural substance, should have a maximum solubility. The preferred wash liquids include especially water and/or alcohols having 1 to 7, preferably 1 to 4 carbon atoms, especially methanol, ethanol, propanol and/or butanol. These liquids may be used individually or else as a mixture of two, three or more liquids.

The temperature in the washing step may likewise be within a wide range, which depends inter alia on the solidification temperature of the encapsulation material. In order to prevent aggregation of the particles, the selected temperature should be below the solidification temperature of the encapsulation material. The temperature is preferably in the range from −20° C. to 100° C., more preferably in the range from −10° C. to 40° C. The pressure used in the washing step is likewise uncritical, and depends in many cases on the type of bioactive natural substance and the solidification temperature of the encapsulation material. For example, the pressure in the washing step may be selected within the range from 10 mbar to 200 bar, preferably within the range from 100 mbar to 100 bar.

The apparatus usable to wash the particles is common knowledge. For example, it is possible for this purpose to use apparatus which comprises a mixing vessel and a separator. The mixing vessels preferably include the units and apparatus for dispersing detailed above.

In a further step, the resulting microparticles may be dried. The apparatus usable to dry the microparticles is common knowledge. For instance, it is possible to use apparatus including drum dryers, tumble dryers, pan dryers, screw dryers, paddle dryers, cylinder dryers, roll dryers, freeze dryers, fluidized bed dryers, spray dryers, flow dryers, grinding dryers, tray dryers, tunnel dryers, vacuum dryers and/or vacuum contact dryers.

The temperature in the drying step may likewise be within a wide range, which depends inter alia on the solidification temperature of the encapsulation material. In order to prevent aggregation of the particles, the selected temperature should be below the solidification temperature of the encapsulation material. The temperature in the drying step is preferably in the range from −20° C. to 50° C., more preferably in the range from −10° C. to 30° C. The pressure used in the drying step is likewise uncritical, and depends in many cases on the type of bioactive ingredient and the solidification temperature of the encapsulation material. For example, the pressure may be selected within the range from 0.1 mbar to 10 bar, preferably in the range from 0.2 mbar to 2 bar.

The process described above can be performed with simple plants which can be constructed from components known per se. Suitable plants preferably comprise at least two mixing vessels and a separator, in which case the mixing vessels are connected to one another via at least one feed and the second mixing vessel is connected to the separator. The second phase removed in the separator can preferably be recycled into a mixing vessel via a recycle line.

In a preferred embodiment, a pump suitable for high-viscosity liquids may be provided in the line between the first mixing vessel in which the melt is produced and the second mixing vessel in which the melt is dispersed in the second liquid phase. The preferred pumps include especially screw pumps, for example screw pumps with one, two or three screws; screw compressors, vane pumps, rotary piston pumps, rotary pumps, piston pumps and/or peristaltic pumps.

In a particular aspect of the present invention, the plant preferably has at least three mixing vessels, in which case at least two mixing vessels are connected to at least one mixing vessel via feeds. In this case, at least one mixing vessel serves to produce the melt, at least one mixing vessel to produce the second liquid phase and at least one mixing vessel to disperse the melt in the second liquid phase. The melt and the second liquid phase may be produced batchwise or continuously in further separate mixing vessels in order to ensure continuous production of microparticles.

The mixing vessels used in the plant for producing inventive preparations may be equipped with temperature control. Accordingly, these mixing vessels may comprise heating elements or cooling elements.

The plant may preferably have at least one dryer connected to the separator. In addition, the plant may preferably comprise an apparatus for washing particles.

The solidification of the melt in the dispersion can be achieved in the plant by means of various measures. For example, it is possible to cool the mixing vessel in which the dispersion has been produced. This can be done, for example, by external cooling or by supplying liquids which preferably have a composition that is the same as or similar to the second liquid phase. For this purpose, it is preferably also possible to use a heat exchanger, a mixing valve or an additional mixing vessel.

The plant may comprise pumps which may serve, for example, for the transport of liquids or for the generation of elevated or reduced pressure. Suitable pumps depend on the particular purpose. The preferred pumps include, for example, positive displacement pumps, such as for example drawing machines, conveying screws, bellows pumps, piston pumps, rotary piston pumps, externally/internally toothed gear pumps, membrane pumps, rotary vane pumps, centrifugal pumps, peristaltic pumps, toothed belt pumps, eccentric spiral pumps, screw pumps and screw compressors and/or hydraulic rams; flow pumps, such as for example centrifugal pumps, axial pumps, diagonal pumps and/or radial pumps; bubble pumps, water-jet pumps, vapor-jet pumps, hydraulic rams, horsehead pumps (bottoms pumps); vacuum pumps, such as for example displacer pumps, propellant pumps, molecular pumps, turbomolecular pumps, cryopumps, sorption pumps, oil diffusion pumps.

Such plants are described by way of example in the figures described in detail hereinafter.

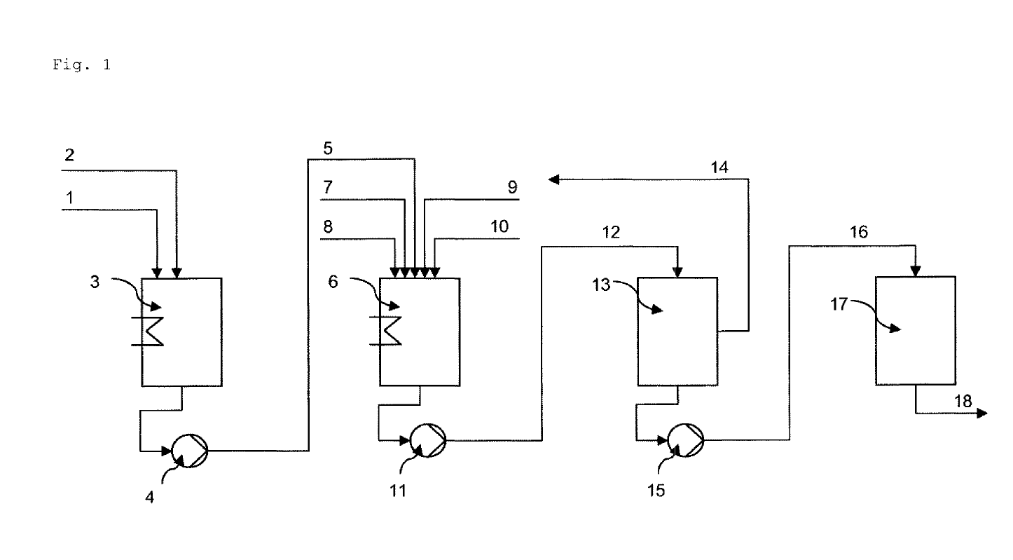

FIG. 1 describes a first embodiment of a plant for performing the process of the present invention.

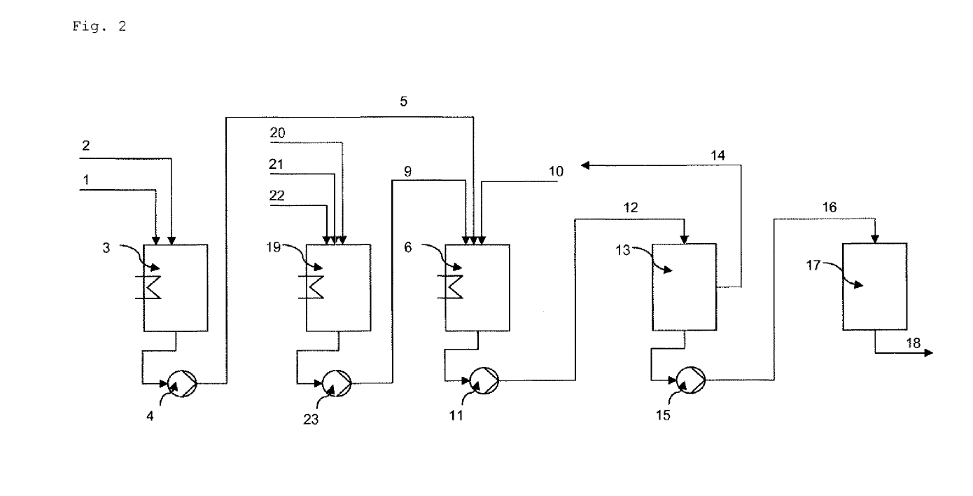

FIG. 2 describes a second embodiment of a plant for performing the process of the present invention.

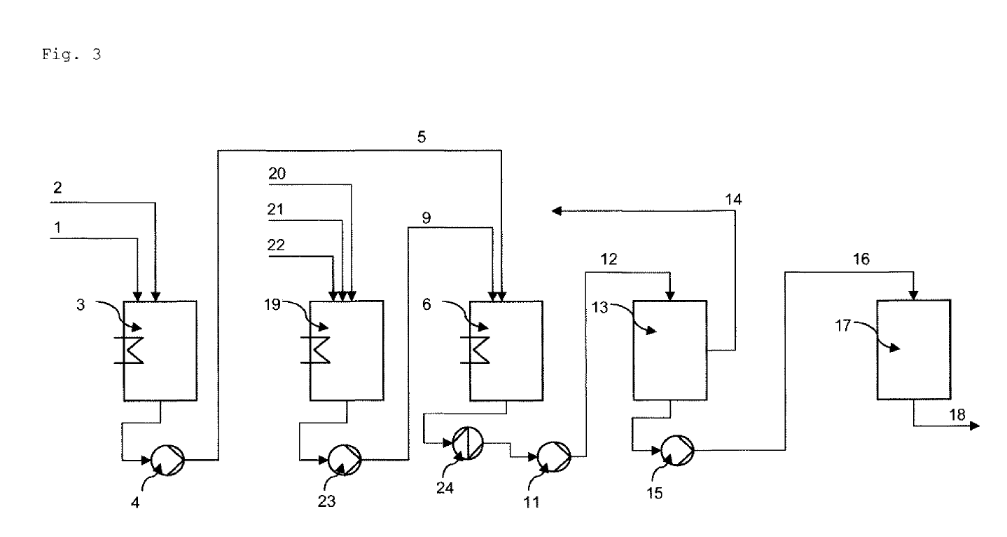

FIG. 3 describes a third embodiment of a plant for performing the process of the present invention.

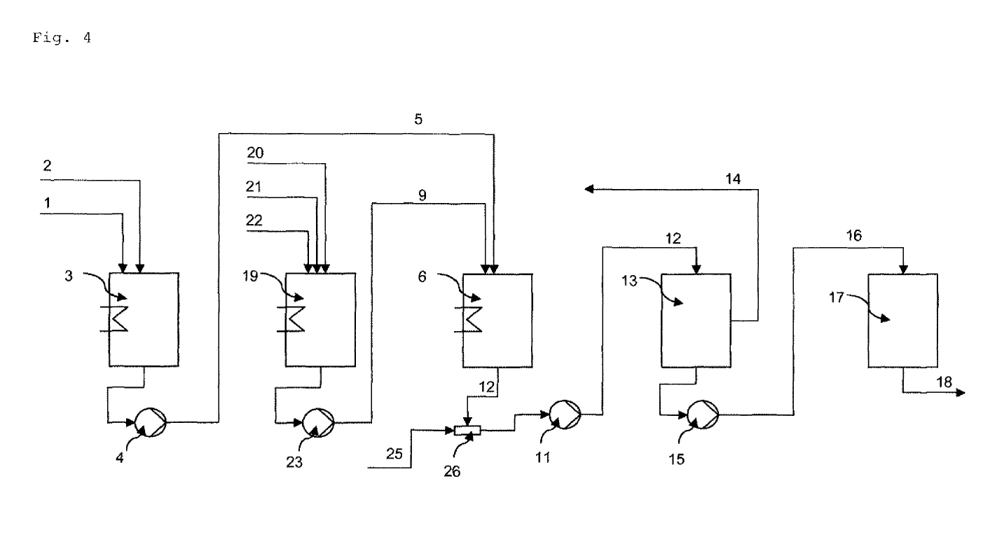

FIG. 4 describes a fourth embodiment of a plant for performing the process of the present invention.

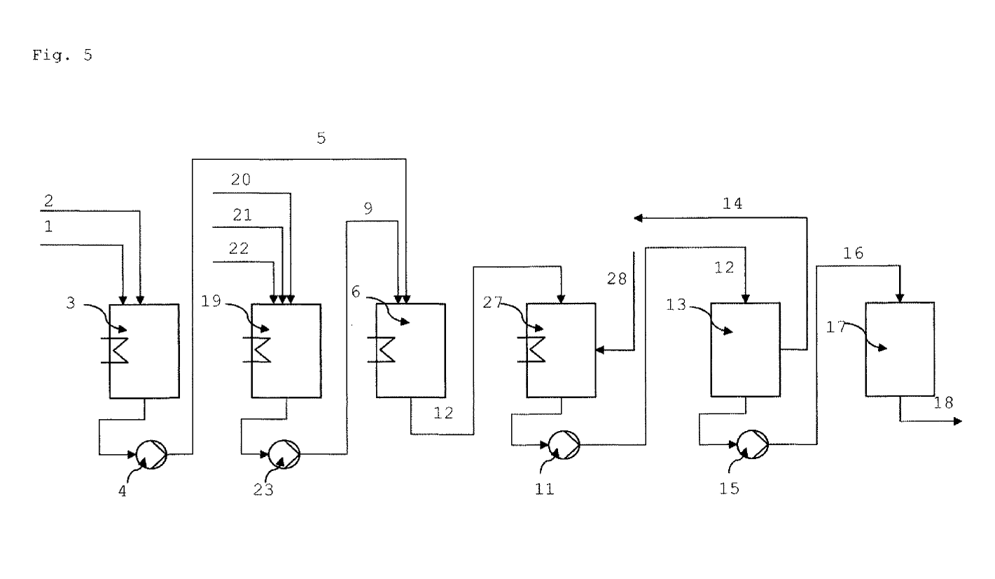

FIG. 5 describes a fifth embodiment of a plant for performing the process of the present invention.

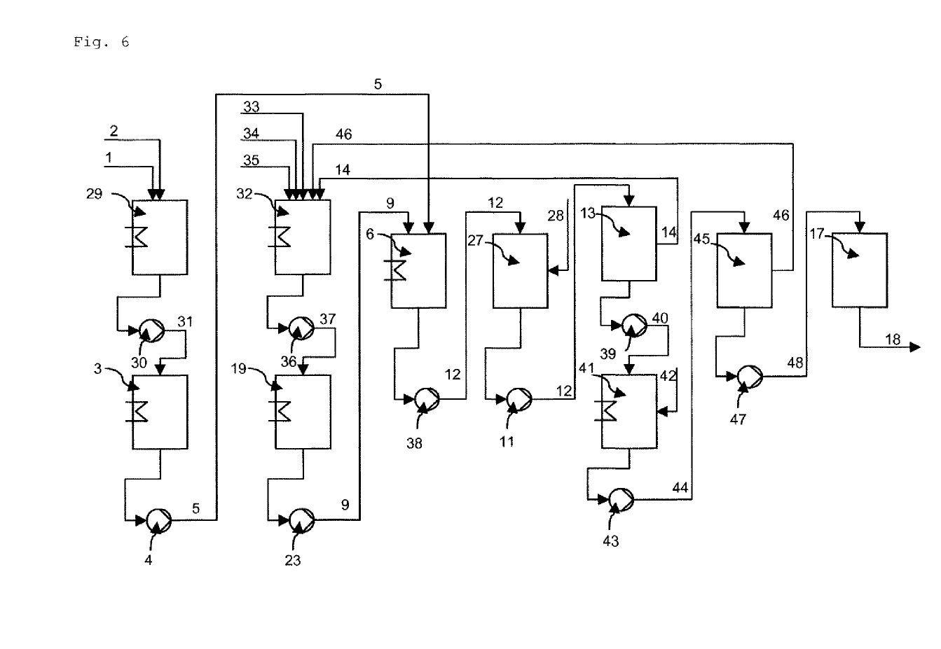

FIG. 6 describes a sixth embodiment of a plant for performing the process of the present invention.

FIG. 1 shows a first embodiment of a plant for performing the process of the present invention. This plant may have, for example, one, two or more feeds 1 and 2, for example conduits or feed screws, by means of which one or more polymers with polyester units, one or more glycerides and/or one or more bioactive natural substances are fed to a first mixing vessel 3. In the mixing vessel 3, the substances fed in can be converted to a melt which comprises at least one polymer with polyester units, at least one glyceride and at least one bioactive natural substance. In the mixing vessel 3, the components can be distributed finely within one another. For example, a solution, a dispersion or a suspension can be prepared. In many cases, the encapsulation material forms the matrix phase in which the bioactive natural substance is distributed. For this purpose, the apparatus described above can be used.

The melt obtained in mixing vessel 3 can be transferred, for example, with a pump 4 by means of the feed 5, for example a conduits, into the mixing vessel 6.

The mixing vessel 6 may have one, two, three, four or more further feeds 7, 8, 9, 10, for example conduits or feed screws, by means of which, for example, stabilizers, emulsifiers, warm water and/or cold water can be fed in. In the present description of the figure, water is used by way of example as the second liquid phase. However, it is obvious to the person skilled in the art that any other compound described above as a main constituent of the second liquid phase can likewise be used instead of or together with water. The water thus serves merely as an example of the compounds detailed above, which can be replaced correspondingly by the other substances.

The feeds 7, 8, 9, 10 can all open into the mixing vessel 6. In addition, these feeds can also be combined upstream of entry into the mixing vessel 6.

Before the feeding of the melt into the mixing vessel 6, it is possible to prepare, for example via feeds 7, 8 and 9, a solution which comprises as the main constituent for example water or ethanol and auxiliaries, for example stabilizers or emulsifiers. This solution can be heated to a temperature above the solidification temperature of the encapsulation material. In addition, the components fed in may already have an appropriate temperature.

After production of a corresponding solution in mixing vessel 6, the melt produced in mixing vessel 3 can be fed to mixing vessel 6. In mixing vessel 6, the melt is dispersed in the solution described above. For this purpose, the mixing vessel 6 has known apparatus for dispersing. For this purpose, the apparatus described above can be used.

Once the desired droplet size and droplet size distribution has been obtained by the dispersing step, the melt present dispersed in the second liquid phase, for example water, can be solidified. To this end, for example, cold water can be introduced into the mixing vessel 6 via a feed 10. In addition, the mixing vessel 6 can be cooled by means of a cooling medium which is passed through a heat exchanger or a jacket.

The particles thus obtained can be separated from the second liquid phase. To this end, the composition obtained in mixing vessel 6, which comprises solidified microparticles, can be transferred into the separator 13, for example with a pump 11 via the conduit 12. The separator 13 serves to separate or concentrate the microparticles present in the second liquid phase, for which any of the apparatus detailed above can be used. In the present case, the microparticles are separated from the second liquid phase in the separator, for which a concentration step may in many cases be sufficient. The separated second liquid phase, which may comprise, for example, water, emulsifiers and stabilizers, can be introduced via a recycle line 14, for example a conduit, into the mixing vessel 6.

The microparticles removed can be transferred into the dryer 17, for example, with a pump 15 via the feed 16, for example a conduit. In the dryer 17, residues of the second liquid phase, for example water, can be removed. The dried microparticles can be withdrawn from the dryer via the conduit 18.

With reference to FIG. 2, a second embodiment of a plant for performing the process of the present invention is described below, which is essentially similar to the first embodiment, and so only the differences are discussed below, the same reference numerals being used for the same parts and the above description applying correspondingly.

Like the first embodiment too, the second embodiment also has a mixing vessel 3 with feeds 1, 2 for producing a melt, a mixing vessel 6 for dispersing the melt in a second liquid phase, a separator 13 and a dryer 17.

In the second embodiment, the second liquid phase is, however, produced in a further mixing vessel 19 which may have, for example, one, two, three or more feeds 20, 21, 22. The feeds 20, 21 and 22 can be used to add the components of the second liquid phase, for example water or ethanol as the main constituent a well as auxiliaries, for example stabilizers and emulsifiers, to the mixing vessel 19. In this example too, the water or the ethanol, independently of the further components, may be replaced by any of the compounds of the second liquid phase detailed above.

The solution obtained in mixing vessel 19 can be transferred into the mixing vessel 6, for example with a pump 23 via the feed 9, for example a conduit. Cold water or cooled ethanol can be introduced into the mixing vessel 6, for example via the feed 10, in order to solidify the dispersed melt. The second liquid phase separated in the separator 13, for example water or ethanol, which may additionally comprise auxiliaries, such as emulsifiers or stabilizers, can be returned into the mixing vessel 6 via recycle line 14. Thus, only the amounts of second liquid phase which cannot be recovered in the separator 13 can be introduced into the mixing vessel 6 from mixing vessel 19.

The further components of the plant correspond essentially to those of the first embodiment, and so reference is made thereto.

With reference to FIG. 3, a third embodiment of a plant for performing the process of the present invention is described hereinafter, which is essentially similar to the second embodiment, and so only the differences are discussed below, the same reference numerals being used for the same parts and the above description applying correspondingly.

Like the second embodiment too, the third embodiment also has a mixing vessel 3 with feeds 1, 2 for producing a melt, a mixing vessel 19 for producing the second liquid phase, a mixing vessel 6 for dispersing the melt in a second liquid phase, a separator 13 and a dryer 17.

In the third embodiment, the cooling of the melt after dispersing it in the second liquid phase is achieved by an external cooling 24, preferably a heat exchanger, which is provided in conduit 12 between the mixing vessel 6 and the separator 13. In the heat exchanger 24, the dispersed melt is solidified.

This particular configuration allows the present process also to be performed continuously. In addition, this embodiment can be operated in a particularly energy-saving manner.

The further components of the plant correspond essentially to those of the second embodiment, and so reference is made thereto.

With reference to FIG. 4, a fourth embodiment of a plant for performing the process of the present invention is described below, which is essentially similar to the second embodiment, and so only the differences are discussed below, the same reference numerals being used for the same parts and the above description applying correspondingly.

Like the second embodiment too, the fourth embodiment also has a mixing vessel 3 with feeds 1, 2 for producing a melt, a mixing vessel 19 for producing the second liquid phase, a mixing vessel 6 for dispersing the melt in a second liquid phase, a separator 13 and a dryer 17.

In the fourth embodiment, the cooling of the melt after dispersing it in the second liquid phase is achieved by feeding in a cold liquid via conduit 25, which corresponds essentially to the composition of the second liquid phase, in order to solidify the melt. The cold liquid can be fed in via a mixing valve 26 which is provided in conduit 12 between the mixing vessel 6 and the separator 13.

A portion of the second liquid phase removed in the separator 13, for example water or ethanol, which may additionally comprise auxiliaries, such as emulsifiers or stabilizers, can be returned into the mixing vessel 6 via the recycle line 14. Thus, only the amounts of second liquid phase which cannot be recovered in the separator 13 can be introduced from mixing vessel 19 into the mixing vessel 6. In this case, this portion can be heated to the temperature of the mixing vessel 6. A further portion of the second liquid phase removed in the separator 13 can be passed into the conduit 25. In this case, the second phase can be cooled, such that the temperature of the second liquid phase introduced into the conduit 25 corresponds to the temperature of the cold liquid.

This particular configuration allows the present process also to be performed continuously. In addition, this embodiment can be operated in a particularly energy-saving manner.

The further components of the plant correspond essentially to those of the second embodiment, and so reference is made thereto.

With reference to FIG. 5, a fifth embodiment of a plant for performing the process of the present invention is described below, which is essentially similar to the second embodiment, and so only the differences are discussed below, the same reference numerals being used for the same parts and the above description applying correspondingly.

Like the second embodiment too, the fifth embodiment also has a mixing vessel 3 with feeds 1, 2 for producing a melt, a mixing vessel 19 for producing the second liquid phase, a mixing vessel 6 for dispersing the melt in a second liquid phase, a separator 13 and a dryer 17.

In the fifth embodiment, the cooling of the melt is carried out after the dispersing step in the second liquid phase in a mixing vessel 27, the cooling being achievable, for example, by feeding in a cold liquid which corresponds essentially to the composition of the second liquid phase, in order to solidify the melt. The cold liquid can be fed in via the conduit 28.

A portion of the second liquid phase removed in the separator 13, for example water or ethanol which may additionally comprise auxiliaries, such as emulsifiers or stabilizers, can be returned into the mixing vessel 6 via the recycle line 14. Thus, only the amounts of second liquid phase which cannot be recovered in the separator 13 can be introduced from mixing vessel 19 into the mixing vessel 6. In this case, this portion can be heated to the temperature of the mixing vessel 6. A further portion of the second liquid phase removed in the separator 13 can be passed into the conduit 28. In this case, the second phase can be cooled, such that the temperature of the second liquid phase introduced into the feed line 28 corresponds to the temperature of the cold liquid.

This particular configuration also allows the present process to be performed continuously. In addition, this embodiment can be operated in a particularly energy-saving manner.

The further components of the plant correspond essentially to those of the second embodiment, and so reference is made thereto.

With reference to FIG. 6, a sixth embodiment of a plant for performing the process of the present invention is described below, which is essentially similar to the fifth embodiment, and so only the differences are discussed below, the same reference numerals being used for the same parts and the above description applying correspondingly.

Like the fifth embodiment too, the sixth embodiment also has a mixing vessel 3 with feeds 1, 2 for producing a melt, a mixing vessel 19 for producing the second liquid phase, a mixing vessel 6 for dispersing the melt in a second liquid phase, a separator 13 and a dryer 17. In this case, the transfer of the composition obtained in the mixing vessel 6 into the mixing vessel 27 can be supported by a pump 38 which is provided in conduit 12.

The sixth embodiment has a mixing vessel 29 in which the melt can be premixed in order to ensure in mixing vessel 3 a fill level, which ensures continuous flow of melt into the mixing vessel 6. In the mixing vessel 29, the melt is formed continuously or in batches, it being possible to feed the encapsulation material and the bioactive natural substance into the mixing vessel 29 via the feeds 1 and 2. The melt can be transferred with a pump 30 via conduit 31 into the mixing vessel 3.

In addition, the second liquid phase can also be preformed in a mixing vessel 32, before the second liquid phase is transferred into the mixing vessel 19. This measure can ensure that second liquid phase is transferred continuously from mixing vessel 19 into the mixing vessel 6. In mixing vessel 32, the second liquid phase can be formed continuously or in batches, and the components can be introduced into the mixing vessel 32 via the feed lines 33, 34 and 35. The second liquid phase produced can be transferred with a pump 36 via conduit 37 into the mixing vessel 19.

A portion of the second liquid phase removed in the separator 13, for example water or ethanol which may additionally comprise auxiliaries, such as emulsifiers or stabilizers, can be returned into the mixing vessel 32 or 19 via the recycle line 14. In addition, a portion of the second liquid phase removed in the separator 13 can be passed via the recycle line into the mixing vessel 6 (not shown). Thus, only the amounts of second liquid phase which cannot be recovered in the separator 13 can be introduced from mixing vessel 19 into the mixing vessel 6. In this case, this portion can be heated to the temperature of the mixing vessel 6 or 32, or 19.

A further portion of the second liquid phase removed in the separator 13 can be passed into the feed line 28. In this case, the second phase can be cooled, such that the temperature of the second liquid phase introduced into the feed line 28 corresponds to the temperature of the cold liquid.

The sixth embodiment has an apparatus for washing the particles. This apparatus comprises a mixing vessel and a separator, and these components can also be accommodated in one housing. Accordingly, the particles separated in separator 13 can be transferred with a pump 39 via feed 40 into a mixing vessel 41 in which the particles can be cleaned with a wash liquid which is supplied via conduit 42. A pump 43 can be used to transfer the composition via conduit 44 into a separator 45. In the separator 45, the cleaned particles are separated from the wash liquid. The wash liquid can be processed and reused, in which case the recycle line 46, according to the type of wash liquid, can be recycled either into the conduit 42 (not shown) or into one of the mixing vessels 6, 19, 27 and/or 32 used previously, in which case the temperature of the wash liquid can be adjusted in each case. The cleaned particles can be transferred with pump 47 via feed 48 into the dryer 17.

This particular configuration also allows the present process to be performed continuously. In addition, this embodiment can be operated in a particularly energy-saving manner. In addition, particles can be obtained with a particularly favorable and controllable release profile.

The further components of the plant correspond essentially to those of the fifth embodiment, and so reference is made thereto.

The inventive preparations can be used, for example, in cosmetics, in medicaments, in deodorants, in foods, in animal feeds, in drinks, in moisture-donating compositions, for example emollients and/or moisturizers, in phytonutrients and/or in packages. The term “emollients” is widespread per se and generally denotes a cosmetic oil which is said to have a moisture-donating property. Some oils of this kind are used to treat dry skin. Phytonutrients are understood to mean especially vegetable-based food additives which have advantageous effects. These food additives may comprise, for example, the above-detailed carotenoids, flavonoids, phytosterols and/or polyphenols.

The present invention will be illustrated hereinafter with reference to examples, without any intention that this should impose a restriction.

### EXAMPLES

### Detection of the Enzymatic Degradation of Boltorn® H30 and Boltorn® H40

The degradation of the Boltorn® H30 and Boltorn® H40 molecules (Perstorp) in aqueous enzyme-containing solutions was demonstrated by the following experiments:

The Boltorn® H30 and Boltorn® H40 polymers (Perstorp) were ground in an electrical mill and sieved in separate tests. The particle fraction between 90 and 250 μm was further employed. The polymer particles were suspended in a solution of lipase from Candida cylindracea, 0.5 mg/ml (Lipase Lipomod® 34P, Biocatalyst Ltd., UK) and phosphate buffer, pH=5, at 37° C. Pure buffer under the same conditions was used as control sample. The concentration of the 2,2-bis(hydroxymethyl)propionic acid monomer of the hyperbranched Boltorn® H30 and Boltorn® H40 polymers was analyzed with the aid of UV spectroscopy (peak at 208.5 nm). The amounts of monomer detected are summarized in tables 1 and 2.

After 24 hours the concentration of the hydroxymethylpropionic acid in the lipase-containing solution is higher by a factor of 4.7 and 4.8 respectively than the concentration in pure buffer. The Boltorn® H30 and Boltorn® H40 polymers are thus enzymatically degradable hyperbranched polymers.

TABLE 1
Degradation of Boltorn ® H30

| | Concentration of hydroxymethylpropionic acid (g/ml) | |
|---|---|---|
| Time in h | Buffer without enzyme | Buffer with enzyme |
| 0 | 0.045 | 0.045 |
| 24 | 0.088 | 0.248 |

TABLE 2
Degradation of Boltorn ® H40

| | Concentration of hydroxymethylpropionic acid (g/ml) | |
|---|---|---|
| Time in h | Buffer without enzyme | Buffer with enzyme |
| 0 | 0.047 | 0.047 |
| 24 | 0.093 | 0.270 |

### Comparative Example 1

### Not According to the Invention

Using the plant shown in FIG. 1, a preparation which comprised kernel oil from peach stones and a hyperbranched polyester was produced.

The hyperbranched polyester used was obtained by hydrophobizing a hydrophilic hyperbranched polyester which had a weight-average molecular weight Mw of 3500 g/mol, a glass transition temperature of about 35° C. and a hydroxyl number of about 490 mg KOH/g (available commercially from Perstorp under the Boltorn® H30 name). The hydrophobization was effected by esterifying the hydrophilic polymer with a mixture of stearic acid and palmitic acid (mass-based ratio of stearic acid to palmitic acid=2 to 1), which converted 90% of the hydroxyl groups of the hydrophilic polymer. The molecular weight MW was 10 000 g/mol. The esterification was performed as described in WO 93/17060. The hydrophobized hyperbranched polyester had a melting point of 49° C.

To produce the preparation, 10% by weight of peach kernel oil was dissolved in the molten polymer at a temperature of about 60° C. with a spiral stirrer at 200 revolutions per minute in a first mixing vessel (vessel with reference 3 in FIG. 1) for 5 minutes.

In a further mixing vessel, a mixture of surfactants consisting of 1% by weight of polyvinyl alcohol (M=6000 g/mol, Polisciences®, Warrington, USA) and 0.1% by weight of an ethoxylated fatty alcohol (Tego® Alkanol L4 from Degussa GmbH) was initially charged in water at 60° C. with stirring. This mixture functions as a continuous phase.

Subsequently, one part by weight of the melt produced in the first mixing vessel, which comprised the kernel oil besides the polymer, was added from the first mixing vessel with continual stirring with an ULTRA-TURRAX® stirrer at 8000 revolutions per minute to 9 parts by weight of the continuous phase into a second mixing vessel (vessel with reference 6 in FIG. 1) at 60° C. After a residence time of 0.2 to 10 minutes and a lowering of the system temperature in the second mixing vessel to a temperature which is 15° C. below the melting temperature of the polymer, particles form. The suspension was fed with a peristaltic pump into a centrifuge, in which the active ingredient particles were separated from the continuous phase at 25° C. and washed with water. Subsequently, the active ingredient particles were dried in a vacuum dryer at 20° C. and 10 mbar for 100 h.

The resulting particles exhibit a particle size distribution of 5 μm<d_{P90}<50 μm and consist of the hyperbranched fatty acid-modified polyester and approx. 9.9% by weight of kernel oil (based on the particle mass).

Subsequently, the properties of the particles were examined. For this purpose, more particularly, the storage stability, the stability of the particles in a buffer solution and the release of the active ingredient by enzymatic degradation were determined.

The storage stability was determined by iodometric titration. To this end, 0.1 g of kernel oil was dispersed in 10 ml of phosphate buffer solution (pH=5) in two different 300 ml flasks (samples KO1 and KO2). The kernel oil in flask KO1 was dissolved with 10 ml of chloroform. Exactly 10 ml of the iodine monobromide reagent solution were pipetted into the solution. The flask was closed, shaken briefly and left to stand in the dark for 1 hour. Subsequently, 20 ml of potassium iodide solution and 100 ml of water were added. The iodine released was back-titrated with sodium thiosulfate solution using starch. After 24 hours, the sample KO2 was likewise titrated by this procedure. The iodine number was calculated with the following equation:

IN=(a−b)·12.69·100/(m·1000)

where
IN=iodine number [g of iodine/100 g of oil]
a=consumption of sodium thiosulfate solution in the blank test [ml]
b=consumption of sodium thiosulfate solution in the particular sample [ml]
m=starting weight of kernel oil [g]

In the same manner, the iodine number was determined for 1 g of the particles from comparative example 1.

The results are compiled in table 3.

TABLE 3
Storage stability of the preparation composed
of kernel oil and hyperbranched polyester

| | Iodine number [g of iodine/100 g of starting weight] | |
|---|---|---|
| | Particles from | |
| Time in h | comparative example 1 | Pure kernel oil |
| 0 | 9.3 | 98.6 |
| 24 | 9.2 | 97.0 |

The release of kernel oil from the particles of comparative example 1 was determined in a buffer without enzyme at pH 5.0 and 37° C. by the iodometric titration detailed above. It was 5.6% after 2 hours and 36.0% after 4 hours, based in each case on the total content of natural substance in the particles. Use of a buffer solution with lipase (Lipomod® 34P; the concentration of the Lipomod 34P was 0.5 mg/ml) increases the aforementioned values to 9.9% after 2 hours and 52.7% after 4 hours.

### Example 1

Comparative example 1 was essentially repeated. However, to produce the preparation, 10% by weight of peach kernel oil were dissolved for 5 minutes at a temperature of about 60° C. with a spiral stirrer at 200 revolutions per minute in a first mixing vessel in a composition which comprises 50% by weight of polymer and 50% by weight of triglyceride. The vegetable-based triglyceride has a melting point of 57-60° C., a density of 0.877 g/cm³ at 60° C. and a dynamic viscosity of 18 mPa·s at 70° C. (commercially available under the Tegin® BL 150 V Roh trade name from Goldschmidt GmbH).

The resulting particles exhibit a particle size distribution of 5 μm<d_{P90}<50 μm and consist of the encapsulation material, which additionally comprises triglyceride besides the hyperbranched fatty acid-modified polyester, and approx. 9.8% by weight of kernel oil (based on the particle mass).

The preparation obtained above exhibited outstanding storage stability. An iodometric titration showed no degradation of the kernel oil after storage for 24 h. Thus, the preparation, immediately after production and after storage at 25° C. for 24 hours, in each case had an iodine number of 9.3 [g of iodine/100 g of formulation].

The release of kernel oil from the particles of example 1 in a buffer at pH 5.0 and 37° C. was 4.8% after 2 hours, 10.3% after 4 hours, based in each case on the total content of natural substance in the particles. Use of a buffer solution comprising lipase (Lipomod 34P; the concentration of the Lipomod 34P was 0.5 mg/ml) increases these values to 11.4% after 2 hours and 56.4% after 4 hours.

It was surprisingly found that the release of the bioactive ingredient from the particles is slowed considerably by the addition of a glyceride with a melting point of at least 35° C., while the enzymatic degradation of the encapsulation material is accelerated. This allows a particularly targeted release of the active ingredient, for example on human skin.

1-32. (canceled)

33. A composition comprising at least one encapsulation material and at least one bioactive natural substance, wherein:
a) said bioactive natural substance can be released from the composition in a controlled manner;
b) said encapsulation material comprises at least one glyceride with a melting point of at least 35° C. and, additionally, at least one polymer with polyester units, wherein said polymer with polyester units has:
i) a melting temperature of at least 30° C.; and
ii) a viscosity in the range of from 50 mPa*s to 250 Pa*s, measured by means of rotational viscometry at 110° C.

34. The composition of claim 33, wherein the bioactive natural substance is dispersed homogeneously in the encapsulation material.

35. The composition of claim 33, wherein the bioactive natural substance is present dissolved in the encapsulation material.

36. The composition of claim 33, wherein said composition has a degree of loading of from 1% to 95%, said degree of loading being given by the proportion by weight of the bioactive natural substance in the total weight of the composition.

37. The composition of claim 33, wherein the polymer with polyester units has a hydroxyl number of from 0 to 200 mg KOH/g.

38. The composition of claim 33, wherein the polymer with polyester units has a melting temperature of at least 35° C.

39. The composition of claim 33, wherein the polymer with polyester units has an acid number of from 0 to 20 mg KOH/g.

40. The composition of claim 33, wherein the polymer with polyester units has a molecular weight in the range from 1000 g/mol to 400 000 g/mol.

41. The composition of claim 33, wherein the polymer with polyester units has a viscosity in the range from 100 mPa*s to 100 Pa*s, measured by means of rotational viscometry at 110° C.

42. The composition of claim 33, wherein the glyceride is a triglyceride.

43. The composition of claim 42, wherein the triglyceride is derived from carboxylic acids having 12 to 24 carbon atoms.

44. The composition of claim 33, wherein the glyceride has a melting point in the range from 45 to 80° C.

45. The composition of claim 33, wherein the glyceride has a dynamic viscosity in the range of from 10 to 50 mPa*s at 70° C.

46. The composition of claim 33, wherein the polymer with polyester units is a hyperbranched polymer comprising a hydrophilic core with polyester units and hydrophobic end groups.

47. The composition of claim 46, wherein the hyperbranched polymer has a molecular weight greater than or equal to 3000 g/mol, a hydroxyl number of from 0 to 200 mg KOH/g, a degree of branching of from 20 to 70% and a melting temperature of at least 30° C.

48. The composition of claim 46, wherein the hyperbranched polymer has a degree of functionalization of at least 5%.

49. The composition of claim 46, wherein the hydrophilic core has at least 90% by weight of repeat units derived from polyester-forming monomers.

50. The composition of claim 46, wherein the hydrophilic core has a central unit which is derived from an initiator molecule having at least two hydroxyl groups, and repeat units which are derived from monomers having at least one carboxyl group and at least two hydroxyl groups.

51. The composition of claim 46, wherein the hydrophobic end groups are formed by groups derived from carboxylic acids having at least 10 carbon atoms.

52. The composition of claim 46, wherein at least some of the hydrophobic end groups are formed by groups derived from carboxylic acids having at most 18 carbon atoms.

53. The composition of claim 52, wherein a proportion of said carboxylic acids have 12 to 18 carbon atoms of at least 30% by weight, based on the weight of the carboxylic acids used for hydrophobization.

54. The composition of claim 33, having a weight ratio of glyceride to polymer with polyester units of from 10:1 to 1:10.

55. The composition of claim 33, wherein the bioactive natural substance comprises at least one compound selected from the group consisting of: tocopherol and derivatives, ascorbic acid and derivatives, deoxyribonucleic acid, retinol and derivatives, alpha-lipoic acid, niacinamide, ubiquinone, bisabolol, allantoin, phytantriol, panthenol, AHA acids, amino acids, hyaluronic acid, polyglutamic acid, beta-glucans, creatin and creatin derivatives, guanidine and guanidine derivatives, ceramides, sphingolipids, phytosphingosine and phytosphingosine derivatives, sphingosine and sphingosine derivatives, sphinganine and sphinganine derivatives, pseudo-ceramides, essential oils, peptides, proteins, protein hydrolysates, plant extracts and vitamin complexes.

56. The composition of claim 33, wherein the bioactive natural substance is a flavoring, an aroma, a natural extract, a flavor-enhancing compound, a natural wax, a protein, a peptide, a vitamin, a vitamin precursor, a fat, a fatty acid, an amino acid, an amino acid precursor, a sugar, a sugar derivative, a nucleotide or a nucleic acid and precursors and derivatives thereof, a medicament, an enzyme, a coenzyme, or a mixture of said compounds.

57. The composition of claim 33, wherein the bioactive natural substance is a kernel oil.

58. The composition of claim 33, wherein the encapsulation material is enzymatically degradable.

59. The composition of claim 33, wherein at least 20% by weight of the bioactive natural substance present in the composition is released upon contact with an enzyme within at most three days.

60. The composition of claim 33, having the form of microparticles with a size of from 1 μm to 1000 μm.

61. The composition of claim 33, wherein the composition is particulate and has at least 80% by weight of particles within a size range from 1 μm to 100 μm.

62. A process for producing a composition as claimed in claim 33, comprising the steps of:
a) producing a melt of an encapsulation material, comprising at least one polymer with polyester units and at least one glyceride with a melting point of at least 35° C., and at least one bioactive natural substance,
b) introducing the melt into a second liquid phase in which the encapsulation material is sparingly soluble, said second liquid phase having a solidification temperature below the solidification temperature of the encapsulation material,
c) dispersing the melt in the second liquid phase at a temperature which is greater than or equal to the solidification temperature of the encapsulation material, and
d) solidifying the melt dispersed in the second liquid phase.

63. The process of claim 62, wherein the encapsulation material has a water solubility at 40° C. of at most 10 percent by mass.
